(19) United States
(12) Patent Application Publication
(10) Pub. No.: US 2024/0254219 A1
(43) Pub. Date: Aug. 1, 2024

(54) NOVEL WNT AGONIST ANTIBODIES AND THERAPEUTIC USES THEREOF

(71) Applicant: The Regents of the University of California, Oakland, CA (US)

(72) Inventors: Bin LIU, San Francisco, CA (US); Nam-Kyung LEE, Daejeon (KR); Scott BIDLINGMAIER, San Francisco, CA (US); Yang SU, South San Francisco, CA (US)

(21) Appl. No.: 18/693,416

(22) PCT Filed: Sep. 20, 2022
(86) PCT No.: PCT/US2022/076706

§ 371 Date: Mar. 19, 2024

Related U.S. Application Data
(60) Provisional application No. 63/246,250, filed on Sep. 20, 2021.

(51) Int. Cl. C07K 16/28; A61K 39/00; A61P 19/10; C12N 15/10

(52) U.S. Cl. CPC C07K 16/28; A61P 19/10; C12N 15/1037; C12N 15/1055; A61K 2039/505; C07K 2317/21; C07K 2317/75

(57) ABSTRACT

Antibodies are provided herein that agonize Wnt signaling, do not compete with a Wnt ligand for LRP6 binding, and activate Wnt signaling in the presence of inhibitors. Methods for promoting cell differentiation and tissue regeneration using the disclosed antibodies are also provided.

### CROSS-REFERENCE TO RELATED APPLICATION

This application claims priority to U.S. Provisional Application No. 63/246,250, filed Sep. 20, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with government support under grants R01 CA118919 and R₀₁ CA171315 awarded by The National Institutes of Health. The government has certain rights in the invention.

### BACKGROUND OF THE INVENTION

The canonical Wnt/β-catenin signaling pathway is involved in various biological processes including tissue regeneration, stem cell regulation, and cell proliferation and differentiation (Clevers et al., Science 346(6205), 1248012 (2014); Lien & Fuchs, Genes & Development 28(14), 1517-32 (2014); Steinhart & Angers, Development 145 (2018)). In particular, the critical role of canonical Wnt signaling in bone formation has been shown by several studies (Baron & Kneissel, Nature Med 19:179-92 (2013); Florio et al., Nature Comm. 12:3247 (2014); Liu et al., Science Transl. Med 8 (2016); McDonald et al., Blood 129:3452-64 (2017); Pozzi et al., Bone 53:487-96 (2013)), including by analysis of the bone degenerative effects of Wnt signaling inhibitors such as sclerostin or Dickkopf Wnt signaling pathway inhibitor 1 (DKK1) (Markham, Drugs 79:471476 (2019)). In particular, sclerostin blockade has shown to be clinically effective against osteoporosis, and an anti-sclerostin monoclonal antibody (romosozumab) has been approved for osteoporosis treatment. In addition, the anti-DKK1 antibody BHQ880 has been clinically evaluated for restoration of osteolytic bone loss caused by multiple myeloma (Fulciniti et al., Blood 114:371-79 (2009); Iyer et al., Brit. J. Haematol. 167:366-75 (2014); Munshi & Anderson, Clin. Canc. Res. 19:333744 (2013)).

Although therapies targeting inhibitory ligands have achieved promising results with respect to promoting bone formation, these methods may be less effective if Wnt ligands are absent or are below a critical threshold in the disease region. In addition, the anti-inhibitor approach is limited to the particular inhibitor that a monoclonal antibody is designed to bind and neutralize. For example, while romosozumab blocks sclerostin, it does not block DKK1, resulting in potentially limited blocking of inhibitory activities toward Wnt signaling (Joiner et al., TEM 24:31-39 (2013)).

Alternatively, Wnt signaling may be directly activated using a canonical Wnt pathway agonist. Canonical Wnt signaling is induced by two distinct Wnt co-receptors, the G protein-coupled receptor Frizzled (Fzd) and the low-density lipoprotein receptor-related protein 5 or 6 (LRP5 or LRP6). Binding of Wnt ligands drives the formation of the Fzd-Wnt-LRP6 complex that leads to LRP6 phosphorylation to initiate the signaling. Inhibition of canonical Wnt signaling by anti-LRP6 antibodies has been reported (Ettenburg et al., PNAS 10:15473-78 (2010)). In addition, a ligand surrogate-based Wnt agonist capable of activating Wnt signaling and promoting bone formation has been reported (Janda et al., Nature 545:234-+ (2017)). This ligand surrogate-based Wnt agonist consists of an anti-Fzd scFv and the DKK1 LRP6-binding domain, thereby mimicking the mechanism of natural Wnt ligands. Another ligand surrogate-based Wnt agonist has been reported that consists of an anti-Fzd scFv and an anti-LRP6 single domain antibody (Fowler et al., Nature Comm. 12:3247 (2021)). Other Wnt ligand surrogates have been described that explore multivalency and crosslinking to enhance signaling, a mechanism that may also be used by natural Wnt ligands and co-activators (Chen et al., Cell. Signal. 26:1068-74 (2014); Tao et al., eLife 8 (2019)). However, because all of the ligand surrogate-based Wnt agonists compete with endogenous Wnt ligands for binding to the receptor complex, they are also subject to inhibition by endogenous inhibitors such as DKK1 and sclerostin that bind to ligand binding sites.

### BRIEF SUMMARY OF THE INVENTION

In various aspects, the inventions disclosed herein may include, but need not be limited to, any one or more of the following embodiments:

In one aspect, the disclosure provides a monoclonal antibody, or an antigen-binding portion thereof, that agonizes Wnt signaling and does not compete with a Wnt ligand or inhibitor.

In another aspect, the disclosure provides a monoclonal antibody, or an antigen-binding portion thereof, that specifically binds to low density lipoprotein receptor-related protein 6 (LRP6) and agonizes Wnt signaling. In some embodiments of this aspect, the antibody or antigen-binding portion comprises a) heavy chain variable domain (VH) comprising (1) the amino acid sequence of SEQ ID NO:2; (2) the amino acid sequence of SEQ ID NO:8; (3) heavy chain CDR1-3 comprising the amino acid sequences of SEQ ID NOs: 18, 19, and 21, respectively; (4) heavy chain CDR1-3 comprising the amino acid sequences of SEQ ID NOs: 18, 19, and 22, respectively; (5) heavy chain CDR1-3 comprising the amino acid sequences of SEQ ID NOs: 18, 19, and 23, respectively; (6) heavy chain CDR1-3 comprising the amino acid sequences of SEQ ID NOs: 18, 19, and 24, respectively; or (7) heavy chain CDR1-3 comprising the amino acid sequences of SEQ ID NOs: 18, 19, and 25, respectively. In some embodiments, the antibody further comprises b) a light chain variable domain (VL) comprising (1) the amino acid sequence of SEQ ID NO:15; (2) light chain CDR1-3 comprising the amino acid sequences of SEQ ID NOs: 26, 27, and 29, respectively; or (3) light chain CDR1-3 comprising the amino acid sequences of SEQ ID NOs: 26, 27, and 30, respectively.

In some embodiments, the monoclonal antibody or antigen-binding portion thereof of claim 1, comprises a VH comprising the amino acid sequence of SEQ ID NO:3 and a VL comprising the amino acid sequence of SEQ ID NO:15. In some embodiments, the antibody comprises a heavy chain variable region having at least 90%, 90%, 91%, 92%, 93%, 94%, 95%, 96%, 97%, 98%, 99%, or higher identity to the amino acid sequence of SEQ ID NO:3. In some embodiments, the antibody comprises a light chain variable region having at least 90%, 90%, 91%, 92%, 93%, 94%, 95%, 96%, 97%, 98%, 99%, or higher identity to the amino acid sequence of SEQ ID NO:15.

In some embodiments, the monoclonal antibody or antigen-binding portion thereof of claim 1, comprises a VH comprising the amino acid sequence of SEQ ID NO:3 and a VL comprising the amino acid sequence of SEQ ID NO:16. In some embodiments, the antibody comprises a heavy chain variable region having at least 90%, 90%, 91%, 92%, 93%, 94%, 95%, 96%, 97%, 98%, 99%, or higher identity to the amino acid sequence of SEQ ID NO:3. In some embodiments, the antibody comprises a light chain variable region having at least 90%, 90%, 91%, 92%, 93%, 94%, 95%, 96%, 97%, 98%, 99%, or higher identity to the amino acid sequence of SEQ ID NO:16.

In some embodiments, the monoclonal antibody or antigen-binding portion thereof of claim 1, comprises a VH comprising the amino acid sequence of SEQ ID NO:9 and a VL comprising the amino acid sequence of SEQ ID NO:16. In some embodiments, the antibody comprises a heavy chain variable region having at least 90%, 90%, 91%, 92%, 93%, 94%, 95%, 96%, 97%, 98%, 99%, or higher identity to the amino acid sequence of SEQ ID NO:9. In some embodiments, the antibody comprises a light chain variable region having at least 90%, 90%, 91%, 92%, 93%, 94%, 95%, 96%, 97%, 98%, 99%, or higher identity to the amino acid sequence of SEQ ID NO:16.

In some embodiments, the monoclonal antibody or antigen-binding portion thereof of claim 1, comprises a VH comprising the amino acid sequence of SEQ ID NO:2 and a VL comprising the amino acid sequence of SEQ ID NO:15. In some embodiments, the antibody comprises a heavy chain variable region having at least 90%, 90%, 91%, 92%, 93%, 94%, 95%, 96%, 97%, 98%, 99%, or higher identity to the amino acid sequence of SEQ ID NO:2. In some embodiments, the antibody comprises a light chain variable region having at least 90%, 90%, 91%, 92%, 93%, 94%, 95%, 96%, 97%, 98%, 99%, or higher identity to the amino acid sequence of SEQ ID NO:15.

In some embodiments, the monoclonal antibody, or an antigen-binding portion thereof comprises a) a light chain comprising the amino acid sequence of SEQ ID NO:15, and b) a heavy chain comprising (1) a heavy chain comprising HCDR1-3 comprising the amino acid sequences of SEQ ID NOs: 18, 19, and 21, respectively; (2) heavy chain CDR1-3 comprising the amino acid sequences of SEQ ID NOs: 18, 19, and 22, respectively; (3) heavy chain CDR1-3 comprising the amino acid sequences of SEQ ID NOs: 18, 19, and 23, respectively; (4) heavy chain CDR1-3 comprising the amino acid sequences of SEQ ID NOs: 18, 19, and 24, respectively; or (5) heavy chain CDR1-3 comprising the amino acid sequences of SEQ ID NOs: 18, 19, and 25, respectively. In certain aspects, the monoclonal antibody or antigen-binding portion thereof comprises a VH comprising the amino acid sequence of SEQ ID NO:3, 4, 5, 6, or 7, and a VL comprising the amino acid sequence of SEQ ID NO:15. In some embodiments, the antibody comprises a heavy chain variable region having at least 90%, 90%, 91%, 92%, 93%, 94%, 95%, 96%, 97%, 98%, 99%, or higher identity to the amino acid sequence of any one of SEQ ID NOS:3-7. In some embodiments, the antibody comprises a light chain variable region having at least 90%, 90%, 91%, 92%, 930, 94%, 95%, 96%, 97%, 98%, 99%, or higher identity to the amino acid sequence of SEQ ID NO:15.

In some embodiments, the monoclonal antibody, or an antigen-binding portion thereof comprises a heavy chain comprising (1) the amino acid sequence of SEQ ID NO:8; (2) heavy chain CDR1-3 comprising the amino acid sequences of SEQ ID NOs: 18, 19, and 21, respectively; (3) heavy chain CDR1-3 comprising the amino acid sequences of SEQ ID NOs: 18, 19, and 22, respectively; (4) heavy chain CDR1-3 comprising the amino acid sequences of SEQ ID NOs: 18, 19, and 23, respectively; (5) heavy chain CDR1-3 comprising the amino acid sequences of SEQ ID NOs: 18, 19, and 24, respectively; or (6) heavy chain CDR1-3 comprising the amino acid sequences of SEQ ID NOs: 18, 19, and 25, respectively. In some embodiments, the monoclonal antibody, or an antigen-binding portion thereof comprises b) a light chain comprising (1) light chain CDR1-3 comprising the amino acid sequences of SEQ ID NOs: 26, 27, and 29, respectively; or (2) light chain CDR1-3 comprising the amino acid sequences of SEQ ID NOs: 26, 27, and 30, respectively. In certain aspects, the monoclonal antibody or antigen-binding portion thereof, comprises a VH comprising the amino acid sequence of SEQ ID NO:8, 9, 10, 11, 12, or 13, and a VL comprising the amino acid sequence of SEQ ID NO:16 or 17. In some embodiments, the antibody comprises a heavy chain variable region having at least 90%, 90%, 91%, 92%, 93%, 94%, 95%, 96%, 97%, 98%, 99%, or higher identity to the amino acid sequence of any one of SEQ ID NOS:8-13. In some embodiments, the antibody comprises a light chain variable region having at least 90%, 90%, 91%, 92%, 93%, 94%, 95%, 96%, 97%, 98%, 99%, or higher identity to the amino acid sequence of SEQ ID NO:16 or SEQ ID NO:17.

In some embodiments, the monoclonal antibody or antigen-binding portion thereof comprises a human IgG heavy chain constant region. In some embodiments, the monoclonal antibody or antigen-binding portion thereof is an effector-attenuated IgG1 antibody. In certain embodiments, the effector-attenuated IgG1 antibody is an IgG1 antibody comprising a leucine to alanine substitution at positions 234 and 235. In some embodiments, the monoclonal antibody or antigen-binding portion thereof is an IgG2 antibody. In some embodiments, the monoclonal antibody or antigen-binding portion thereof is human.

In some embodiments, the monoclonal antibody or antigen-binding portion thereof specifically binds to an epitope on LRP6 that does not overlap with the binding site for a Wnt ligand or inhibitor. The Wnt ligand may be, for example, Wnt1, Wnt2, Wnt2b, Wnt3, Wnt3a, Wnt8a, Wnt8b, Wnt10a, Wnt10b, Wnts2b, or Wnt9b. The Wnt inhibitor may be, for example, Dickkopf Wnt signaling pathway inhibitor 1 (DKK1), Dickkopf Wnt signaling pathway inhibitor 2 (DKK2), Dickkopf Wnt signaling pathway inhibitor 3 (DKK3), Dickkopf Wnt signaling pathway inhibitor 4 (DKK4), Dickkopf Like Acrosomal Protein 1 (DKKL1), sclerostin (SOST), Wise (SOSTDC1 (sclerostin domain-containing 1)), IGFBP-4, or Waif1/5T4. In some embodiments, the monoclonal antibody or antigen-binding portion binds to a non-linear epitope. In some embodiments, the epitope comprises K662 and K684. In some embodiments, the epitope does not include E663, E708, H834, Y875, or M877.

In another aspect, the disclosure provides a pharmaceutical composition comprising the Wnt agonist antibody or antigen-binding portion thereof and a pharmaceutically acceptable excipient.

In another aspect, nucleic acid sequences encoding the Wnt agonist antibody or antigen-binding portion thereof are provided. The disclosure also describes vectors and mammalian host cells comprising the nucleic acid sequences. In some embodiments, the host cell is a CHO, CHO-K1, CHO-S, ExpiCHO, CHO-DG44, CHO-Pro minus, HEK293A, HEK293F cell. In some embodiments, the disclosure provides methods for producing the monoclonal antibody or antigen-binding portion thereof, comprising culturing the host cell under conditions to allow for production of the monoclonal antibody or antigen-binding portion thereof.

In yet another aspect, the disclosure provides methods for promoting tissue regeneration, comprising adding the Wnt agonist monoclonal antibody or antigen-binding portion thereof described herein to a cell or tissue in vitro or ex vivo.

In another aspect, the disclosure provides methods for restoring tissue in an individual in need thereof, comprising administering to the individual the pharmaceutical composition comprising the Wnt agonist antibody or antigen-binding portion thereof described herein. In some embodiments, the tissue is bone tissue, intestine tissue, liver tissue, or brain tissue. In some embodiments, the individual has a disease or condition characterized by insufficient Wnt signaling. In some embodiments, the individual has age-induced osteoporosis, drug induced bone loss, osteogenesis imperfecta, inflammatory bowel disease, severe alcoholic hepatitis, diabetic retinopathy, wet age-related macular degeneration (AMD), Fuchs' dystrophy, limbal stem cell deficiency, dry AMD, Sjögren's dry eye, short bowel syndrome, or hearing loss. In some embodiments, the pharmaceutical composition comprising the Wnt agonist antibody or antigen-binding portion thereof described herein is administered by intravenous injection, intraperitoneal injection, or subcutaneous injection.

In another aspect, the disclosure provides methods for identifying a monoclonal antibody or an antigen-binding portion thereof that agonizes Wnt signaling and does not compete with a Wnt ligand or inhibitor, comprising: a) providing an LRP6 polypeptide or a portion thereof comprising at least the LRP6 polypeptide P3E3P4E4 domain; b) contacting the LRP6 polypeptide or portion thereof with a library of binding molecules; c) selecting one or more binding molecules from the library that bind to the LRP6 polypeptide or portion thereof; and d) identifying selected binding molecules that do not compete with a Wnt ligand or inhibitor for binding to the LRP6 polypeptide or portion thereof.

### BRIEF DESCRIPTION OF THE DRAWINGS

FIGS. 1A-1H demonstrate the binding of an exemplary Wnt-agonist human monoclonal antibody 66 (shown as 6-6 in the figures) to LRP6. FIG. 1A and FIG. 1B are graphs showing the activation of canonical Wnt signaling by 100 nM of the Wnt-agonist antibody in the presence of Wnt1 or Wnt3a (FIG. 1A) or in the absence of an endogenous Wnt ligand (FIG. 1B) (Error bars represent SD for n=2. **P<0.01; ***P<0.001.).

FIG. 1C is a schematic showing deletion constructs of LRP6 that were used in binding assays (SP: Signal peptide; P3 and P4: Beta-propeller domain 3 and 4, respectively; E3 and E4: EGF-like domain 3 and 4, respectively; LDLR: Low-density lipoprotein receptor type A domain; TM: Transmembrane domain; Cyto: cytoplasmic domain).

The results of the binding assays are shown in FIG. 1D. Each LRP6 truncation plasmid was separately transfected into HEK293 cells with a GFP-expressing construct. Binding of the 66 IgG in GFP-positive cell population was analyzed by flow cytometry.

FIG. 1E is a graph showing the effect of the 66 antibody on activation of Wnt signaling on cells expressing truncated LRP6. LRP6 truncation constructs and STF reporter plasmids were transfected into HEK293 cells, and the cells were incubated in Wnt3aCM with or without 66 IgG. Error bars represent SD for n=2. *P<0.05, ***P<0.001. N.S=not significant.

FIG. 1F is a graph showing fine epitope mapping by alanine scanning of LRP6 and the effect of the alanine substitutions on binding of the 66 antibody. LRP6 single mutants and the double mutant (K662A/K684A) were separately transfected into HEK293 cells. An anti-LRP6 scFv-Fc fusion that binds to the LRP6-P1 domain was used as a control to confirm cell surface expression of LRP6. Binding of the 66 antibody was determined by flow cytometry, and median fluorescence intensity (MFI) values were normalized against MFI of the P1-binding scFv-Fc.

FIG. 1G is a graph showing the effect of an LRP6 double mutant (K662A/K684A) on binding of the 66 antibody in the presence or absence of the Wnt3a ligand. A plasmid encoding the wild-type LRP6 (WT), or the K662A/K684A double mutant was transfected with Wnt3a-expression plasmid and STF reporter expression plasmid into HEK293 cells, and the cells were incubated with or without 66 IgG. Error bars represent SD for n=2. *P<0.05.

FIG. 1H shows the structure of the LRP6 double mutant (K662A/K684A), with the residues involved in binding to the Wnt3a ligand shown in yellow (E663, E708, H834, Y875, M877) and the residues involved in binding to the 66 antibody (K662A and K684) shown in red.

FIG. 2A and FIG. 2B are graphs showing the binding kinetics of the exemplary Wnt-agonist human monoclonal antibody 66 (shown as 6-6 in the figures) with the Wnt3a ligand and/or the inhibitor DKK1. Recombinant Wnt3a (FIG. 2A) or DKK1 (FIG. 2B) was allowed to bind to LRP6-loaded biosensors. The biosensors were further dipped in a mixture of Wnt3a and DKK1 (black), Wnt3a and 66 Fab (blue in D), or DKK1 and 66 Fab (red in FIG. 2B). The data show that the 66 antibody binds to LRP6 in the presence of either Wnt3a or DKK1.

FIG. 2C is a graph showing the effects of the inhibitor DKK1 on the agonist activity of the 66 antibody. HEK293 cells were transfected with the STF reporter and Wnt3a-expression constructs. The cells were incubated with or without the 66 IgG (50 nM) and DKK1 (20 nM). Error bars represent SD for n=2.

FIG. 2D is a schematic diagram showing that the exemplary Wnt-agonist human monoclonal antibody (66) acts as a new type of Wnt ligand that has an additive effect to endogenous ligands and activates Wnt signaling in the presence of inhibitors.

FIG. 3A shows that DKK1 inhibits Wnt3a/RSPO2-induced β-catenin signaling. HEK293 cells were transfected with the STF reporter and Wnt3a-expression construct and incubated with RSPO2 only (5 nM) or RSPO2 (5 nM) plus DKK1 (15 nM). Error bars represent SD for n=2. The design and results are schematically summarized in the right panel.

FIG. 3B shows that DKK1 has no inhibitory effect on 66/RSPO2-induced Wnt/β-catenin signaling. HEK293 cells transfected with the STF reporter constructs were incubated with 66 (100 nM), RSPO2 (5 nM), or DKK1 (15 nM) as indicated. No Wnt ligands were added. Error bars represent SD for n=2. The design and results are schematically summarized in the right panel.

FIG. 3C and FIG. 3D show that RSPO2-induced Wnt/0-catenin signaling is enhanced by the 66 antibody. In FIG. 3C, RSPO2 was titrated on HEK293 cells transfected with the STF reporter and Wnt3a-expression constructs. A constant concentration of 66 IgG (20 nM) was added in culture medium. In FIG. 3D, HEK293 cells were transfected with the STF reporter and Wnt3a expression construct, and incubated with varying concentrations of 66 IgG in the presence of RSPO2 (5 nM). Error bars represent SD for n=2. The design and results are schematically summarized in the right panel.

FIG. 4 shows that the exemplary Wnt-agonist human monoclonal antibody 66 (shown as 6-6 in the figures) promotes osteoblast differentiation in vitro. FIG. 4A is a graph showing the cross-species binding of 66 to the extracellular domain of recombinant human or mouse LRP6 in ELISA assays. Ctrl IgG: a non-binding human IgG. Error bars represent SD for n=2.

FIG. 4B and FIG. 4C are graphs showing Wnt/β-catenin signaling enhancement by 66 IgG in mouse cell lines MC3T3-E1 (FIG. 4B) or C3H/10T1/2 (FIG. 4C). MC3T3-E1 (FIG. 4B) or C3H/10T1/2 (FIG. 4C) cell line was transfected with Wnt3a-expression and STF reporter constructs and further incubated with or without the 66 IgG. Luciferase activity was normalized against a control group transfected with the reporter construct only. Data represent mean f SD for n=2. *P<0.05.

FIG. 4D shows graphs that demonstrate relative mRNA expression for osteoblast marker genes (Runx2, BMP2, ALP, and OCN) detected by qRT-PCR. C3H/10T1/2 cells were incubated for 3 days with Wnt3aCM or 66 IgG as indicated. Expression of osteoblast marker genes (RUNX2, BMP2, ALP, and OCN) was assessed by qRT-PCR. Relative mRNA expression levels were calculated using the comparative Ct method and normalized to GAPDH gene. *P<0.05; **P<0.01.

FIG. 4E is a graph showing ALP activity induced by the Wnt-agonist 66 antibody in the presence or absence of Wnt3a conditioned media. C3H/10T1/2 cells were cultured in Wnt3aCM with or without 66 IgG for 7 days. Cell lysates were used to measure ALP activity, which was normalized against a control group without Wnt3aCM and 66 treatment. Error bars represent SD (n=2). *P<0.05.

FIG. 4F shows the relative mineralization induced by Wnt3a conditioned media in the presence or absence of the Wnt-agonist 66 antibody (shown graphically (left panel) or with Alizarin Red staining (right panel)). C3H/10T1/2 cells were cultured for 21 days in osteogenic medium supplemented with Wnt3aCM or 66 IgG as indicated. Alizarin Red staining assay was applied to quantify mineralization (e/), which was normalized against the control (Wnt3aCM-/Ab-). The measurement was presented as mean±SD (n=2). *P<0.05. Representative images were shown (right). Scale bar: 200 μm.

FIG. 5 shows that the exemplary Wnt-agonist human monoclonal antibody 66 (shown as 6-6 in the figures) overcomes multiple myeloma-mediated Wnt signaling inhibition. FIG. 5A is a graph showing inhibition of Wnt3a/β-catenin signaling by culture media from three multiple myeloma cell lines. HEK293 cells transfected with the STF reporter and Wnt3a-expression constructs were incubated in conditioned media (CM) obtained from multiple myeloma cell lines and HEK293 (as control, Ctrl-CM). Values represent mean±SD for n=2. *P<0.05, **P<0.01.

FIG. 5B is a graph showing that the 66 antibody overcomes Wnt signaling inhibition brought on by MM1.S-cultured media. HEK293 cells were transfected with the STF reporter and Wnt3a-expression constructs and incubated in MM1.S-CM with varying concentrations of 66 IgG. Values represent mean f SD (n=2). *P<0.05, **P<0.01.

FIG. 5C is a schematic diagram of an animal study in which the 66 antibody is used in weekly dosing after MM1.S cells were intrafemorally injected in the right femur and were allowed to establish for 1 week. A total of 6 weekly intraperitoneal injections of PBS or 66 IgG (10 mg/kg) were given (n=5/group). Femurs from live mice were scanned by micro-computed tomography 1 week after termination of dosing. A week after n vivo scan, mice were sacrificed, and serum and femur tissues were collected for further analysis.

FIG. 5D is a graph showing the results of ELISA assays evaluating human Ig-lambda light chain concentration in serum of mice with MM1.S implantation that were naïve or injected with PBS or the 66 antibody (Naïve: mice with MM1.S implantation. PBS: mice with MM1.S implantation injected with PBS (vehicle control). 66: mice with MM1.S implantation injected with 66 IgG. N.S. denotes no significance. ***P<0.001).

FIG. 5E shows planar and 3D views of whole femur obtained from micro-CT of these mice. Threshold of micro-CT images were optimized to generate clear planar sections (top) and further reconstructed to obtain stacked 3D views (bottom).

FIG. 6 shows that the exemplary Wnt-agonist human monoclonal antibody 66 (shown as 6-6 in the figures) reverses bone loss in the intrafemoral MM1.S model. FIG. 6A shows a 3D view of trabecular and cortical bone regions of interest in femurs. Micro-CT images were used to reconstruct 3D data set of trabecular and cortical bone regions as indicated.

FIG. 6B shows representative images of the trabecular ROI of naïve mice or mice with MM1.S-implanted femurs (with PBS or the 66 antibody). The micro-CT images of the naïve or MM1.S-implanted femurs (PBS and 66 IgG) were utilized to reconstruct 3D architectures of trabecular bone regions. Representative images were shown.

FIG. 6C and FIG. 6D are graphs showing the quantification of trabecular bone micro-architectures, including bone volume over tissue volume (BV/TV; FIG. 6C) and trabecular bone thickness (FIG. 6D). *P<0.05.

FIG. 6E shows that the 66 antibody enhanced cortical bone formation, as shown visually by micro-CT images (left panel) and graphically as a measure of cortical bone thickness (right panel). Micro-CT images of cortical bone were reconstructed from proximal femur regions (left). Cortical bone thickness (Ct.Th) was measured and compared between the groups (right). *P<0.05.

FIG. 6F shows the histological evaluation of osteoblasts in distal femur regions, with the left panel showing hematoxylin and eosin staining (small panel indicated in the left image is enlarged in the right image, with yellow triangles identifying osteoblasts) and the right panel showing graphically as a measure of the number of osteoblasts on the trabecular bone lining. Femurs with and without antibody treatment were subjected to H&E staining (left). Scale bar=50 μm. Yellow arrowheads indicate osteoblasts, and osteoblast number (Ob.N) on trabecular bone lining was counted (right). *P<0.05.

FIG. 7 shows that the 66 antibody (6-6 in the figures) binds to a different site on LRP6 than previously identified LRP6 binders. HEK293 cells were transfected with LRP6 expression plasmid and incubated with 66 or E34N19 scFv-phage for 1 hour. E34N19 was identified previously as a Wnt antagonist binding to the P3E3P4E4 domain (Lee et al., 2018). The E34N19 IgG was simultaneously added as a competitor. Bound phages were detected by sequential incubation of mouse anti-fd IgG and PE-labeled anti-mouse IgG. FIG. 7A is a graph showing the dissociation curve of the 66 antibody. HEK293 cells were incubated with varying concentrations of 66 IgG, and the binding was analyzed by flow cytometry. Apparent K_{D} (˜5.0 nM) was estimated by curve fitting. FIG. 7B is a graph showing as a measure of mean fluorescence intensity that in a binding study, the E34N19E IgG does not compete with the 66 scFv-phage. As a positive control, the E34N19E IgG competes with the E34N19E scFv-phage.

FIG. 8A and FIG. 8B show modeling of the 66 antibody (shown as 6-6 in the figures) using the Rosetta Antibody module and its interaction with the LRP6-P3E3 domain (PDB:3S8Z in FIG. 8A and PDB:4A0P) using structural docking by ZDOCK. (Blue: CDRHs of 66 Fv, Light grey: LRP6-P3E3 domain, purple: Predicted potential binding sites (within 4 angstrom) by 66 Fv in S2A (T659, G660, K662. L683, K684, T685, H698, V699, E701. F702, G703, D735, G736, Q737, H738, R739) or in S2B (T659, G660, V661, K662, S682, L683, K684, T685, 5687, H698, V699, V700, E701, F702, D735, G736, Q737, H738, R739). Yellow: Residues involved in Wnt3a binding (E663, E708, H834, Y875, M877)) (Chen et al., 2011).

FIG. 8C is a graph showing that the inhibitor DKK1 does not inhibit 66-induced Wnt/β-catenin signaling in the absence of Wnt ligands. HEK293 cells were transfected with the STF reporter constructs and incubated with 66 (100 nM) with or without DKK1 (15 nM). No Wnt ligands were added. Error bars represent SD for n=2. *P<0.05.

FIG. 8D is a graph showing that the 66 antibody enhances Wnt/β-catenin signaling in the absence of Wnt ligands. The 66 antibody was titrated on HEK293 cells transfected with the STF reporter constructs without Wnt ligands. EC50 (13.38 nM) was estimated by curve fitting. Error bars represent SD for n=2.

FIG. 9 is a graph demonstrating that the 66 antibody (shown as 6-6 in the figure) synergizes Wnt/β-catenin signaling amplification with Wnt ligands and RSPO2. HEK293 cells were transfected with the STF reporter and Wnt1 expression construct, and incubated with varying concentrations of 66 IgG in the presence of RSPO2 (5 nM). Error bars represent SD for n=2.

FIG. 10 is a photograph showing the immunohistochemical staining of the distal region of femur tissue obtained from mice that were treated with PBS or the 66 antibody with an anti-human Ig-lambda light chain antibody to mark MM1.S cells. Femur tissues obtained from PBS- or 66-treated mice were formalin-fixed and paraffin embedded, sectioned at 4 μm and stained with anti-human Ig-lambda (λ) light chain antibody to mark MM1.S cells and counter-stained with hematoxylin. The left panel is of femur tissue with no tumor implantation, and the right panel is of femur tissue that was MM1.S injected. Scale bar=100 μm.

FIG. 11 is a schematic diagram of an experiment conducted to analyze the effect of exemplary Wnt agonist antibodies (66 and 66-11) in an ovariectomy-induced osteoporosis mouse model system. The Wnt agonist antibody (6 mg/kg or 1 mg/kg) or PBS control was delivered intraperitoneally or subcutaneously to ovariectomized mice once weekly for 5 weeks as indicated, starting 4 weeks post ovariectomy. In vivo micro-CT scans were performed, focusing on the distal femur bone at the indicated time points. Both trabecular and cortical bone were evaluated.

FIG. 12 is a graph showing the results of micro-CT scans of trabecular bone from an experiment conducted as shown in the diagram of FIG. 11. The micro-CT scans were performed at 4 days after the last dose of Wnt agonist antibody or PBS control was administered. The y axis shows the trabecular bone volume over the total volume, and the x axis shows what was administered and the route of administration. Six mice were used per study group on the x axis, and both legs were scanned to generate images for analysis by ImageJ (BoneJ). The 66 antibody was administered intraperitoneally at 6 mg/kg. The 66-11 antibody was administered intraperitoneally or subcutaneously at 1 mg/kg. * P<0.05; *** P<0.001.

FIG. 13A and FIG. 13B show the results of micro-CT scans of trabecular bone from an experiment conducted as shown in the diagram of FIG. 11. The micro-CT scans were performed at 28 days after the last dose of Wnt agonist antibody or PBS control was administered. FIG. 13A shows the results in a graph. The y axis shows the trabecular bone volume over the total volume, and the x axis shows what was administered and the route of administration. Six mice were used per study group on the x axis, and both legs were scanned. The 66 antibody was administered intraperitoneally at 6 mg/kg. The 66-11 antibody was administered intraperitoneally or subcutaneously at 1 mg/kg. ** P<0.01; *** P<0.001. FIG. 13B shows exemplary images used for analysis of each study group by ImageJ (BoneJ).

FIG. 14A and FIG. 14B show the results of micro-CT scans of trabecular bone from an experiment conducted as shown in the diagram of FIG. 11. The micro-CT scans were performed at 73 days after the last dose of Wnt agonist antibody or PBS control was administered. FIG. 14A shows the results in a graph. The y axis shows the trabecular bone volume over the total volume, and the x axis shows what was administered and the route of administration. Six mice were used per study group on the x axis, and both legs were scanned. The 66 antibody was administered intraperitoneally at 6 mg/kg. The 66-11 antibody was administered intraperitoneally or subcutaneously at 1 mg/kg. ** P<0.01: *** P<0.001. FIG. 14B shows exemplary images used for analysis by ImageJ (BoneJ) of the PBS control study group and the study group treated subcutaneously with the 66-11 antibody.

FIG. 15 is a graph showing the results of micro-CT scans of trabecular bone from an experiment conducted as shown in the diagram of FIG. 11. The micro-CT scans were performed at 111 days after the last dose of Wnt agonist antibody or PBS control was administered. The y axis shows the trabecular bone volume over the total volume, and the x axis shows what was administered and the route of administration. Six mice were used per study group on the x axis, and both legs were scanned to generate images for analysis by ImageJ (BoneJ). The 66 antibody was administered intraperitoneally at 6 mg/kg. The 66-11 antibody was administered intraperitoneally or subcutaneously at 1 mg/kg. ** P<0.01; **** P<0.0001.

FIG. 16 is a graph showing the results of micro-CT scans of cortical bone from an experiment conducted as shown in the diagram of FIG. 11. The micro-CT scans were performed at 4 days after the last dose of Wnt agonist antibody or PBS control was administered. The y axis shows the cortical bone thickness (Ct.Th) in μm, and the x axis shows what was administered and the route of administration. Six mice were used per study group on the x axis, and both legs were scanned to generate images for analysis by ImageJ (BoneJ). The 66 antibody was administered intraperitoneally at 6 mg/kg. The 66-11 antibody was administered intraperitoneally or subcutaneously at 1 mg/kg. * P<0.05: ns: not significant.

FIG. 17 is a graph showing the results of micro-CT scans of cortical bone from an experiment conducted as shown in the diagram of FIG. 11. The micro-CT scans were performed at 28 days after the last dose of Wnt agonist antibody or PBS control was administered. The y axis shows the cortical bone thickness (Ct.Th) in μm, and the x axis shows what was administered and the route of administration. Six mice were used per study group on the x axis, and both legs were scanned to generate images for analysis by ImageJ (BoneJ). The 66 antibody was administered intraperitoneally at 6 mg/kg. The 66-11 antibody was administered intraperitoneally or subcutaneously at 1 mg/kg. * P<0.05: ns: not significant.

FIG. 18 is a graph showing the results of micro-CT scans of cortical bone from an experiment conducted as shown in the diagram of FIG. 11. The micro-CT scans were performed at 73 days after the last dose of Wnt agonist antibody or PBS control was administered. The y axis shows the cortical bone thickness (Ct.Th) in μm, and the x axis shows what was administered and the route of administration. Six mice were used per study group on the x axis, and both legs were scanned to generate images for analysis by ImageJ (BoneJ). The 66 antibody was administered intraperitoneally at 6 mg/kg. The 66-11 antibody was administered intraperitoneally or subcutaneously at 1 mg/kg. *** P<0.001; ns: not significant.

FIG. 19 is a graph showing the results of micro-CT scans of cortical bone from an experiment conducted as shown in the diagram of FIG. 11. The micro-CT scans were performed at 111 days after the last dose of Wnt agonist antibody or PBS control was administered. The y axis shows the cortical bone thickness (Ct.Th) in μm, and the x axis shows what was administered and the route of administration. Six mice were used per study group on the x axis, and both legs were scanned to generate images for analysis by ImageJ (BoneJ). The 66 antibody was administered intraperitoneally at 6 mg/kg. The 66-11 antibody was administered intraperitoneally or subcutaneously at 1 mg/kg. ** P<0.01: ns: not significant.

### DETAILED DESCRIPTION OF THE INVENTION

### I. Introduction

Antibodies are described herein that agonize the canonical Wnt pathway but that do not act as a surrogate for known Wnt ligands. These novel antibodies can activate canonical Wnt signaling in the absence of endogenous Wnt ligands, and the activation is further amplified by R-spondin. In addition, the agonist activity of these antibodies is not blocked by endogenous inhibitors such as DKK1 and sclerostin. These novel agonist antibodies can be used to promote tissue regeneration. For example, the agonist antibodies may be used to activate canonical Wnt/β-catenin signaling to promote cell differentiation or tissue regeneration in vitro or ex vivo and for the treatment of tissue loss (e.g., bone, intestine, liver, brain tissue) and other degenerative conditions caused by disease or aging.

### II. Definitions

As used in herein, the singular forms “a.” “an,” and “the” include plural referents unless the content clearly dictates otherwise. Thus, for example, reference to “an antibody” optionally includes a combination of two or more such molecules, and the like.

A “Wnt agonist” refers to an agent that increases the canonical Wnt/0-catenin signaling pathway, thereby promoting, for example, tissue regeneration and cell differentiation See. e.g., (Clevers et al., Science 346, 54-+(2014); Lien & Fuchs, Genes & Development 28, 1517-1532 (2014): Steinhart & Angers, Development 145 (2018)).

The terms “polypeptide,” “peptide” and “protein” are used interchangeably herein to refer to a polymer of amino acid residues. The terms encompass to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An antibody as described herein can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, the antibody is IgG (e.g., IgG1, IgG2, IgG3, IgG4), IgM, IgA, IgD, or IgE.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

The term “antibody” as used herein includes antibody fragments that retain binding specificity. For example, there are a number of well characterized antibody fragments. Thus, for example, pepsin digests an antibody C-terminal to the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see. Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

In an antibody, substitution variants have at least one amino acid residue removed and a different residue inserted in its place. The sites of greatest interest for substitutional mutagenesis include the hypervariable regions, but framework alterations are also contemplated. Examples of conservative substitutions are described above.

Substantial modifications in the biological properties of the antibody are accomplished by selecting substitutions that differ significantly in their effect on maintaining (a) the structure of the polypeptide backbone in the area of the substitution, for example, as a β-sheet or helical conformation. (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile:
(2) Polar without charge: Cys, Ser, Thr, Asn, Gin;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.
   Non-conservative substitutions are made by exchanging a member of one of these classes for another class.

One type of substitution that can be made is to change one or more cysteines in the antibody, which may be chemically reactive, to another residue, such as, without limitation, alanine or serine. For example, there can be a substitution of a non-canonical cysteine. The substitution can be made in a complementary determining region (CDR) or framework region of a variable domain or in the constant region of an antibody. In some embodiments, the cysteine is canonical (e.g., involved in di-sulfide bond formation). Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Wnt agonist antibodies for promoting tissue regeneration and for treating tissue loss (e.g., loss of bone tissue, intestinal tissue, liver tissue, or brain tissue) also include heavy chain dimers, such as antibodies from camelids. In some embodiments an antibody is dimeric. In other embodiments, the antibody may be in a monomeric form that has an active isotype. In some embodiments the antibody is in a multivalent form, e.g., a trivalent or tetravalent form.

As used herein, the terms “variable region” and “variable domain” refer to the portions of the light and heavy chains of an antibody that include amino acid sequences of complementary determining regions (CDRs, e.g., HCDR1, HCDR2, HCR3, LCDR1, LCDR2, and LCDR3) and framework regions (FRs). The variable region for the heavy and light chains is commonly designated V_{H} and V_{L}, respectively. The variable region is included on Fab, F(ab′)₂, Fv, and scFv antibody fragments described herein, and involved in specific antigen recognition.

As used herein, “complementarity-determining region” or “CDR” refers to the three hypervariable regions in each chain that interrupt the four framework regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

Unless indicated otherwise, the CDR1, CDR2, and CDR3 of the heavy chain variable regions and the CDR1, CDR2, and CDR3 of the light chain variable regions as discussed here are determined by the North method. (see. e.g., North et al., J. Mol. Biol. 406(2):228-256, 2011). In some embodiments, the antibody comprises the CDR1, CDR2, and CDR3, as determined by the North method, of the heavy and light chain variable regions of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17. North's method was developed using a dataset of antibody structures that was fifteen-fold larger than the set used in developing the Chothia numbering scheme. In defining the boundary positions for the CDRs, the North method selected positions that 1) had little structural variability across antibodies (the anchors of each CDR loop (the residue immediately before or after the loop) contain tightly clustered conformations relative to the framework) and 2) were across from each other in the β-sheet framework (i.e., extending equal lengths into the framework). North defined CDRs such that they were more or less symmetric between the VH and VL domains.

In other embodiments, the CDRs of an antibody can be determined using other various well-known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, Structural repertoire of the human V_{H} segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin et al., Methods Enzymol., 203, 121-153, (1991); Pedersen et al., Immunomethods, 1. 126, (1992); and Rees et al., In Sternberg M. J. E. (ed.), Protein Structure Prediction. Oxford University Press, Oxford, 141-172 1996).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci USA 91: 969, 1994).

The terms “antigen,” “immunogen,” “antibody target,” “target analyte,” and like terms are used herein to refer to a molecule, compound, or complex that is recognized by an antibody, i.e., can be specifically bound by the antibody. The term can refer to any molecule that can be specifically recognized by an antibody, e.g., a polypeptide, polynucleotide, carbohydrate, lipid, chemical moiety, or combinations thereof (e.g., phosphorylated or glycosylated polypeptides, etc.). One of ordinary skill in the art will understand that the term does not indicate that the molecule is immunogenic in every context, but simply indicates that it can be targeted by an antibody.

Antibodies bind to an “epitope” on an antigen. The epitope is the localized site on the antigen that is recognized and bound by the antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. &e. e.g., Epitope Mapping Protocols in Methods in Molecular Biology, Vol. 66, Glenn E. Morris, Ed (1996).

A “label” or a “detectable moiety” is a diagnostic agent or component detectable by spectroscopic, radiological, photochemical, biochemical, immunochemical, chemical, or other physical means. Exemplary labels include radiolabels (e.g., ¹¹¹In, ^{99m}Tc, ¹³¹I, ⁶⁷Ga) and other FDA-approved imaging agents. Additional labels include ³²P, fluorescent dyes, electron-dense reagents, enzymes, biotin, digoxigenin, or haptens and proteins or other entities which can be made detectable, e.g., by incorporating a radiolabel into the targeting agent. Any method known in the art for conjugating a nucleic acid or nanocarrier to the label may be employed. e.g., using methods described in Hermanson, Bioconjugate Techniques 1996, Academic Press. Inc., San Diego.

A “labeled” or “tagged” antibody or agent is one that is bound, either covalently, through a linker or a chemical bond, or noncovalently, through ionic, van der Waals, electrostatic, or hydrogen bonds to a label such that the presence of the antibody or agent may be detected by detecting the presence of the label bound to the antibody or agent.

Techniques for conjugating detectable and therapeutic agents to antibodies are well known (see, e.g., Amon et al., “Monoclonal Antibodies For Immunotargeting Of Drugs In Cancer Therapy.” in Monoclonal Antibodies And Cancer Therapy. Reisfeld et al. (eds.), pp. 243-56 (Alan R. Liss, Inc. 1985): Hellstrom et al., “Antibodies For Drug Delivery” in Controlled Drug Delivery (2nd Ed.), Robinson et al. (eds.), pp. 623-53 (Marcel Dekker, Inc. 1987): Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates,” Immunol. Rev., 62:119-58 (1982)).

The terms “specific for,” “specifically binds,” and like terms refer to a molecule (e.g., antibody or antibody fragment) that binds to a target with at least 2-fold greater affinity than non-target compounds, e.g., at least any of 2-fold, 3-fold, 4-fold, 5-fold, 6-fold, 7-fold, 8-fold, 9-fold, 10-fold, 20-fold, 25-fold, 50-fold, 100-fold, 1,000-fold, 10,000-fold, or greater affinity for the target compared to an unrelated target when assayed under the same binding affinity assay conditions. For example, an antibody that specifically binds a target (e.g., human or murine LRP6) will typically bind the target with at least a 2-fold greater affinity than a non-target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity). In certain embodiments, the term “specific binding,” “specifically binds to,” or “is specific for” a particular target, as used herein, can be exhibited, for example, by a molecule (e.g., an antibody) having an equilibrium dissociation constant K_{D} for the target of, e.g., 10⁻² M or smaller, e.g., 10⁻³ M, 10⁻⁴ M, 10⁻⁵ M, 10⁻⁶ M, 10⁻⁷ M, 10⁻⁸ M, 10⁻⁹M, 10⁻¹⁰ M, 10⁻¹¹ M, or 10⁻¹² M. In some embodiments, an antibody has a K_{D} of less than 100 nM or less than 10 nM.

The term “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” As described below, the preferred algorithms can account for gaps and the like. Preferably, identity exists over a region that is at least about 25 amino acids or nucleotides in length, or more preferably over a region that is 50-100 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from about 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. BLAST and BLAST 2.0 are used, with the parameters described herein, to determine percent sequence identity for the nucleic acids and proteins of the disclosure. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached The BLAST algorithm parameters W, T. and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value or a range gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of ordinary skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “therapeutically effective dose,” “effective dose,” or “therapeutically effective amount” herein is meant a dose that produces effects for which it is administered. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of therapeutic effect of at least any 5%, at least 10%, at least 15%, at least 20%, at least 25%, at least 40%, at least 50%, at least 60%, at least 75%, at least 80%, at least 90%, or at least 100%. Therapeutic efficacy can also be expressed as “-fold” increase or decrease. For example, a therapeutically effective amount can have at least 1.2-fold, at least 1.5-fold, at least 2-fold, at least 5-fold, or more effect over a control.

As used here, the term “pharmaceutically acceptable carrier” an excipient or diluent in a pharmaceutical composition. The pharmaceutically acceptable carrier must be compatible with the other ingredients of the formulation and not deleterious to the recipient. In some embodiments, the pharmaceutically acceptable carrier must provide adequate pharmaceutical stability to the active ingredient. The nature of the carrier differs with the mode of administration. For example, for intravenous administration, an aqueous solution carrier is generally used; for oral administration, a solid carrier is preferred.

The term “agonize,” “agonizing,” or the like, when used in the context of agonizing the canonical Wnt/β-catenin signaling pathway refers to any detectable positive change or increase in quantity of a parameter that reflects Wnt signaling, compared to a standard value obtained under the same conditions but in the absence of an antibody as described herein (e.g., Wnt agonist antibodies). The level of this increase following exposure to an antibody as described herein is, in some embodiments, at least 10%, at least 20%, at least 30%, at least 40%, at least 50%, at least 60%, at least 70%, at least 80%, at least 90%, at least 95%, or 100%.

The term “compete,” as used herein with regard to an antibody, means that a first antibody, or an antigen-binding portion thereof, competes for binding with a second antibody, or an antigen-binding portion thereof, or a ligand or inhibitor, where binding of the first antibody with its cognate epitope is detectably decreased in the presence of the second antibody, ligand, or inhibitor compared to the binding of the first antibody in the absence of the second antibody, ligand, or inhibitor. The alternative, where the binding of the second antibody, ligand, or inhibitor to its epitope is also detectably decreased in the presence of the first antibody, can, but need not be the case. That is, a first antibody can inhibit the binding of a second antibody, ligand, or inhibitor to its epitope without that second antibody, ligand, or inhibitor inhibiting the binding of the first antibody to its respective epitope. However, where each antibody, ligand, or inhibitor detectably inhibits the binding of the other antibody, ligand, or inhibitor with its cognate epitope or ligand, whether to the same, greater, or lesser extent, the antibodies are said to “cross-compete” with each other for binding of their respective epitope(s). Both competing and cross-competing antibodies are encompassed by the present disclosure. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

Numerous types of competitive binding assays are known, for example: solid phase direct or indirect radioimmunoassay (RIA), solid phase direct or indirect enzyme immunoassay (EIA), sandwich competition assay (see Stabli et al., Methods in Enzymology 9:242-253 (1983)); solid phase direct biotin-avidin EIA (see Kirkland el al., J. Immunol. 137:3614-3619 (1986)); solid phase direct labeled assay, solid phase direct labeled sandwich assay (see Harlow and Lane, Antibodies, A Laboratory Manual, Cold Spring Harbor Press (1988)); solid phase direct label RIA using I-125 label (see Morel et al.,Molec. Immunol. 25(1):7-15 (1988)); solid phase direct biotin-avidin EIA (Cheung et al., Virology 176:546-552(1990)); and direct labeled RIA (Moldenhauer et al., Scand. J. Immunol. 32:77-82 (1990)). Typically, such an assay involves the use of purified antigen bound to a solid surface or cells bearing either of these, an unlabelled test immunoglobulin and a labeled reference immunoglobulin. Competitive inhibition is measured by determining the amount of label bound to the solid surface or cells in the presence of the test immunoglobulin. Usually, the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

The term “treat” and “treatment” are used herein to refer to both therapeutic treatment and prophylactic or preventive measures, wherein the object is to prevent or slow down an undesired physiological change or disorder. For purpose of this disclosure, beneficial or desired clinical results include, but are not limited to, decreasing tissue loss, promoting cell differentiation or tissue regeneration, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

### III. Wnt Agonist Antibodies

Antibodies (including antibody fragments) that agonize the Wnt/β-catenin signaling pathway are provided. These agonist antibodies specifically bind to LRP6 and can be used to treat or prevent tissue loss are provided. The human LRP6 amino acid sequence can be found at Uniprot accession number O75581. The mouse LRP6 amino acid sequence can be found at Uniprot accession number O88572.

In some embodiments, the monoclonal antibody or antigen-binding portion thereof specifically binds to an epitope on LRP6 that does not overlap with the binding site for a known Wnt ligand or inhibitor. The Wnt ligand may be, for example, Wnt1, Wnt2, Wnt2b (Wnt13), Wnt3, Wnt3a, Wnt4, Wnt5a, Wnt5b, Wnt6, Wnt7a, Wnt7b, Wnt8a, Wnt8b, Wnt9a (Wnt14). Wnt9b (Wnt14b), Wnt10a, Wnt10b, Wnt11, or Wnt16. In certain embodiments, the Wnt ligand is involved in the canonical signaling pathway (e.g., Wnt1, Wnt2, Wnt2b, Wnt3, Wnt3a, Wnt8a, Wnt8b. Wnt10a, Wnt10b, Wnts2b, and Wnt9b). The Wnt inhibitor may be, for example, Dickkopf Wnt signaling pathway inhibitor 1 (DKK1), Dickkopf Wnt signaling pathway inhibitor 2 (DKK2), Dickkopf Wnt signaling pathway inhibitor 3 (DKK3), Dickkopf Wnt signaling pathway inhibitor 4 (DKK4), Dickkopf Like Acrosomal Protein 1 (DKKL1), sclerostin (SOST), Wise (SOSTDC1 (sclerostin domain-containing 1)), IGFBP-4, or Waif1/5T4. In some embodiments, the monoclonal antibody or antigen-binding portion binds to a non-linear epitope. In some embodiments, the monoclonal antibody or antigen-binding portion binds to the P3 domain of LRP6. In some embodiments, the epitope comprises K662 and K684. In some embodiments, the epitope does not include E663, E708, H834, Y875, or M877.

In some embodiments, the disclosed Wnt agonist antibodies comprise sequences of a heavy chain complementary determining region 1 (HCDR1), an HCDR2, an HCDR3, a light chain complementary determining region 1 (LCDR1), a LCDR2, a LCDR3, a heavy chain variable region (VH), and/or a light chain variable region (VL) as described in Tables 1 and 2. The CDRs described in Tables 1 and 2 were determined by the North method (see. e.g., North et al., J. Mol. Biol. 406(2):228-256, 2011).

TABLE 1

| Seq# | Name | VH sequence | Seq# | CDR sequence |
|---|---|---|---|---|
| 1 | 66VH | QVQLLQSGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 20 | AKEGPNSGYFDFDY |
| | | ADSVKGRFTISRDNYKNMLYL | | |
| | | QMNSLRAEDTAVYYCAKEGP | | |
| | | NSGYFDFDYWGQGTLVTVSS | | |
| 2 | 66germ VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 20 | AKEGPNSGYFDFDY |
| | | ADSVKGRFTISRDNSKNTLYL | | |
| | | QMNSLRAEDTAVYYCAKEGP | | |
| | | NSGYFDFDYWGQGTLVTVSS | | |
| 3 | 66_11VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 21 | AKESPFSAYFTFDY |
| | | ADSVKGRFTISRDNSKNTLYL | | |
| | | QMNSLRAEDTAVYYCAKESP | | |
| | | FSAYFTFDYWGQGTLVTVSS | | |
| 4 | 66_11_1VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 22 | AKEGPNSGYFTFDY |
| | | ADSVKGRFTISRDNSKNTLYL | | |
| | | QMNSLRAEDTAVYYCAKEGP | | |
| | | NSGYFTFDYWGQGTLVTVSS | | |
| 5 | 66_11_2VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 23 | AKEGPFSGYFTFDY |
| | | ADSVKGRFTISRDNSKNTLYL | | |
| | | QMNSLRAEDTAVYYCAKEGP | | |
| | | FSGYFTFDYWGQGTLVTVSS | | |
| 6 | 66_11_3VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 24 | AKEGPYSGYFTFDY |
| | | ADSVKGRFTISRDNSKNTLYL | | |
| | | QMNSLRAEDTAVYYCAKEGP | | |
| | | YSGYFTFDYWGQGTLVTVSS | | |
| 7 | 66_11_4VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 25 | AKESPYSAYFTFDY |
| | | ADSVKGRFTISRDNSKNTLYL | | |
| | | QMNSLRAEDTAVYYCAKESP | | |
| | | YSAYFTFDYWGQGTLVTVSS | | |
| 8 | 66germ VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 20 | AKEGPNSGYFDFDY |
| | | SVKGRFTISRDNSKNTLYLQM | | |
| | | NSLRAEDTAVYYCAKEGPNS | | |
| | | GYFDFDYWGQGTLVTVSS | | |
| 9 | 66_11VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 21 | AKESPFSAYFTFDY |
| | | SVKGRFTISRDNSKNTLYLQM | | |
| | | NSLRAEDTAVYYCAKESPESA | | |
| | | YFTFDYWGQGTLVTVSS | | |
| 10 | 66_11_1VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 22 | AKEGPNSGYFTEDY |
| | | SVKGRFTISRDNSKNTLYLQM | | |
| | | NSLRAEDTAVYYCAKEGPNS | | |
| | | GYFTFDYWGQGTLVTVSS | | |
| 11 | 66_11_2VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 23 | AKEGPFSGYFTFDY |
| | | SVKGRFTISRDNSKNTLYLQM | | |
| | | NSLRAEDTAVYYCAKEGPFSG | | |
| | | YFTFDYWGQGTLVTVSS | | |
| 12 | 66_11_3VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 24 | AKEGPYSGYFTFDY |
| | | SVKGRFTISRDNSKNTLYLQM | | |
| | | NSLRAEDTAVYYCAKEGPYS | | |
| | | GYFTFDYWGQGTLVTVSS | | |
| 13 | 66_11_4VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 25 | AKESPYSAYFTFDY |
| | | SVKGRFTISRDNSKNTLYLQM | | |
| | | NSLRAEDTAVYYCAKESPYSA | | |
| | | YFTFDYWGQGTLVTVSS | | |

TABLE 2

| Seq# | Name | VL sequence | Seq# | CDR Sequence |
|---|---|---|---|---|
| 14 | 66VL | EIVLTQSPSSLSASVGDRVTITC | 26 | RASQSISTYLN |
| | | RASQSISTYLNWYQQKPGKAP | 27 | YAASSLQS |
| | | KVLIYAASSLQSGVPSRISGSG | 28 | QQSYSIPLT |
| | | SGTDFTLTISSLQPEDFATYYC | | |
| | | QQSYSIPLTFGGGTKLEIK | | |
| 15 | 66germ VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 28 | QQSYSIPLT |
| | | GSGTDFTLTISSLQPEDFATYY | | |
| | | CQQSYSIPLTFGQGTKVEIK | | |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |
| 17 | 66_11_2VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 30 | QQSYSPPLT |
| | | GSGTDFTLTISSLQPEDFATYY | | |
| | | CQQSYSPPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 3 | 66_11VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 21 | AKESPFSAYFTFDY |
| | | ADSVKGRFTISRDNSKNTLYL | | |
| | | QMNSLRAEDTAVYYCAKESP | | |
| | | FSAYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 15 | 66germ VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 28 | QQSYSIPLT |
| | | GSGTDFTLTISSLQPEDFATYY | | |
| | | CQQSYSIPLTFGQGTKVEIK | | |

In some embodiments, the antibody is the 66-11 antibody.

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 3 | 66_11VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 21 | AKESPFSAYFTFDY |
| | | ADSVKGRFTISRDNSKNTLYL | | |
| | | QMNSLRAEDTAVYYCAKESP | | |
| | | FSAYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 9 | 66_11VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 21 | AKESPFSAYFTFDY |
| | | SVKGRFTISRDNSKNTLYLQM | | |
| | | NSLRAEDTAVYYCAKESPFSA | | |
| | | YFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below: SEQ Name Sequence SEQ CDR Sequence

| SEQ | | | SEQ | |
|---|---|---|---|---|
| ID | Name | Sequence | ID | CDR Sequence |
| 2 | 66germ VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 20 | AKEGPNSGYFDFDY |
| | | ADSVKGRFTISRDNSKNTLYL | | |
| | | QMNSLRAEDTAVYYCAKEGP | | |
| | | NSGYFDFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 15 | 66germ VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 28 | QQSYSIPLT |
| | | GSGTDFTLTISSLQPEDFATYY | | |
| | | CQQSYSIPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 4 | 66 11 1VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 22 | AKEGPNSGYFTFDY |
| | | ADSVKGRFTISRDNSKNTLYL | | |
| | | QMNSLRAEDTAVYYCAKEGP | | |
| | | NSGYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 15 | 66germ VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 28 | QQSYSIPLT |
| | | GSGTDFTLTISSLQPEDFATYY | | |
| | | CQQSYSIPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 5 | 66_11_2VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 23 | AKEGPFSGYFTFDY |
| | | ADSVKGRFTISRDNSKNTLYL | | |
| | | QMNSLRAEDTAVYYCAKEGP | | |
| | | FSGYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 15 | 66germ VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 28 | QQSYSIPLT |
| | | GSGTDFTLTISSLQPEDFATYY | | |
| | | CQQSYSIPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 6 | 66_11_3VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 24 | AKEGPYSGYFTFDY |
| | | ADSVKGRFTISRDNSKNTLYL | | |
| | | QMNSLRAEDTAVYYCAKEGP | | |
| | | YSGYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 15 | 66germ VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 28 | QQSYSIPLT |
| | | GSGTDFTLTISSLQPEDFATYY | | |
| | | CQQSYSIPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 7 | 66_11_4VH | EVQLLESGGGLVQPGGSLRLS | 18 | AASGFTFSTYAMS |
| | | CAASGFTFSTYAMSWVRQAP | 19 | TIGPSGSSTY |
| | | GKGLEWVSTIGPSGSSTYYAY | 25 | AKESPYSAYFTFDY |
| | | ADSVKGRFTISRDNSKNTLYL | | |
| | | QMNSLRAEDTAVYYCAKESP | | |
| | | YSAYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 15 | 66germ VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 28 | QQSYSIPLT |
| | | GSGTDFTLTISSLQPEDFATYY | | |
| | | CQQSYSIPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 8 | 66germ VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 20 | AKEGPNSGYFDFDY |
| | | SVKGRFTISRDNSKNTLYLQM | | |
| | | NSLRAEDTAVYYCAKEGPNS | | |
| | | GYFDFDYWGQGTLVTVSS | | |

In combination with the light chain variable region comprising the CDRs of, or the entire light chain variable sequence, displayed below;

| SEQ | | | SEQ | |
|---|---|---|---|---|
| ID | Name | Sequence | ID | CDR Sequence |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 10 | 66_11_1VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 22 | AKEGPNSGYFTFDY |
| | | SVKGRFTISRDNSKNTLYLQM | | |
| | | NSLRAEDTAVYYCAKEGPNS | | |
| | | GYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 11 | 66_11_2VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 23 | AKEGPFSGYFTFDY |
| | | SVKGRFTISRDNSKNTLYLQM | | |
| | | NSLRAEDTAVYYCAKEGPFSG | | |
| | | YFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 12 | 66_11_3VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 24 | AKEGPYSGYFTFDY |
| | | SVKGRFTISRDNSKNTLYLQM | | |
| | | NSLRAEDTAVYYCAKEGPYS | | |
| | | GYFTFDYWGQGTLVTVSS | | |

In combination with the light chain variable region comprising the CDRs of, or the entire light chain variable sequence, displayed below;

| SEQ | | | SEQ | |
|---|---|---|---|---|
| ID | Name | Sequence | ID | CDR Sequence |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 13 | 66_11_4VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 25 | AKESPYSAYFTFDY |
| | | SVKGRFTISRDNSKNTLYLQM | | |
| | | NSLRAEDTAVYYCAKESPYSA | | |
| | | YFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 16 | 66_11_1VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 29 | QQSYSRPLT |
| | | GSGTDFTLTISSLQPEDFATYY | | |
| | | CQQSYSRPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 8 | 66germ VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 20 | AKEGPNSGYFDFDY |
| | | SVKGRFTISRDNSKNTLYLQM | | |
| | | NSLRAEDTAVYYCAKEGPNS | | |
| | | GYFDFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 17 | 66_11_2VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 30 | QQSYSPPLT |
| | | GSGTDFTLTISSLQPEDFATYY | | |
| | | CQQSYSPPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 9 | 66_11VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 21 | AKESPFSAYFTFDY |
| | | SVKGRFTISRDNSKNTLYLQM | | |
| | | NSLRAEDTAVYYCAKESPESA | | |
| | | YFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 17 | 66_11_2VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 30 | QQSYSPPLT |
| | | GSGTDFTLTISSLQPEDFATYY | | |
| | | CQQSYSPPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 10 | 66_11_1VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 22 | AKEGPNSGYFTFDY |
| | | SVKGRFTISRDNSKNTLYLQM | | |
| | | NSLRAEDTAVYYCAKEGPNS | | |
| | | GYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 17 | 66_11_2VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 30 | QQSYSPPLT |
| | | GSGTDFTLTISSLQPEDFATYY | | |
| | | CQQSYSPPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 11 | 66_11_2VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 23 | AKEGPFSGYFTFDY |
| | | SVKGRFTISRDNSKNTLYLQM | | |
| | | NSLRAEDTAVYYCAKEGPFSG | | |
| | | YFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 17 | 66_11_2VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 30 | QQSYSPPLT |
| | | GSGTDFTLTISSLQPEDFATYY | | |
| | | CQQSYSPPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 12 | 66_11_3VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 24 | AKEGPYSGYFTFDY |
| | | SVKGRFTISRDNSKNTLYLQM | | |
| | | NSLRAEDTAVYYCAKEGPYS | | |
| | | GYFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 17 | 66_11_2VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 30 | QQSYSPPLT |
| | | GSGTDFTLTISSLQPEDFATYY | | |
| | | CQQSYSPPLTFGQGTKVEIK | | |

In some embodiments, an antibody described herein comprises a variable region that specifically binds to LRP6, wherein the heavy chain variable region comprises the CDRs of, or the entire heavy chain variable sequence, displayed below:

| SEQ | | | SEQ | |
|---|---|---|---|---|
| ID | Name | Sequence | ID | CDR Sequence |
| 13 | 66_11_4VH- | EVQLLESGGGLVQPGGSLRLS | 18 | AASGFTFSTYAMS |
| | YA | CAASGFTFSTYAMSWVRQAP | 19 | TIGPSGSSTY |
| | | GKGLEWVSTIGPSGSSTYYAD | 25 | AKESPYSAYFTFDY |
| | | SVKGRFTISRDNSKNTLYLQM | | |
| | | NSLRAEDTAVYYCAKESPYSA | | |
| | | YFTFDYWGQGTLVTVSS | | |

In combination with the light chain variable region comprising the CDRs of, or the entire light chain variable sequence, displayed below:

| SEQ | | | SEQ | |
|---|---|---|---|---|
| ID | Name | Sequence | ID | CDR Sequence |
| 17 | 66_11_2VL | DIQMTQSPSSLSASVGDRVTIT | 26 | RASQSISTYLN |
| | | CRASQSISTYLNWYQQKPGKA | 27 | YAASSLQS |
| | | PKLLIYAASSLQSGVPSRFSGS | 30 | QQSYSPPLT |
| | | GSGTDFTLTISSLQPEDFATYY | | |
| | | CQQSYSPPLTFGQGTKVEIK | | |

In some embodiments, the antibody comprises a heavy chain variable region having at least 90% identity (e.g., 90%, 91%, 92%, 93%, 94%, 95%, 96%, 97%, 98%, 99%, or 100%) to the amino acid sequence of any one of SEQ ID NOS:3-7. In some embodiments, the antibody comprises a light chain variable region having at least 90% identity (e.g., 90%, 91%, 92%, 93%, 94%, 95%, 96%, 97%, 98%, 99%, or 100%) to the amino acid sequence of SEQ ID NO:15.

In some embodiments, the antibody comprises a heavy chain variable region having at least 90% identity (e.g., 90%, 91%, 92%, 93%, 94%, 95%, 96%, 97%, 98%, 99%, or 100%) to the amino acid sequence of any one of SEQ ID NOS:8-13. In some embodiments, the antibody comprises a light chain variable region having at least 90% identity (e.g., 90%, 91%, 92%, 93%, 94%, 95%, 96%, 97%, 98%, 99%, or 100%) to the amino acid sequence of SEQ ID NO:16 or SEQ ID NO:17.

Any of the Wnt agonist antibodies described herein can include one or more human framework regions (e.g., 1, 2, 3, or 4 FRs). In some embodiments, the one or more human framework regions include at least one back mutation.

In further embodiments, a Wnt agonist antibody described herein can cross-react with mouse LRP6. In certain embodiments, the Wnt agonist antibody agonizes canonical Wnt/1-catenin signaling pathway. In addition, the Wnt agonist antibody does not compete with a Wnt ligand or inhibitor for LRP6 binding. Moreover, the Wnt agonist antibody can activate the canonical Wnt pathway in the presence of Wnt inhibitors and/or the absence of Wnt ligands and can amplify the signaling Wnt/β-catenin signaling in the presence of RSPO2.

In some embodiments, a modification can optionally be introduced into the antibodies (e.g., within the polypeptide chain or at either the N- or C-terminal), e.g., to extend in vivo half-life, such as PEGylation or incorporation of long-chain polyethylene glycol polymers (PEG). Introduction of PEG or long chain polymers of PEG increases the effective molecular weight of the polypeptides, for example, to prevent rapid filtration into the urine. In some embodiments, a Lysine residue in the sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are known in the art (described, for example, in Veronese. F. M., et al., Drug Disc. Today 10: 1451-8 (2005): Greenwald, R. B., et al., Adv. Drug Deliv. Rev. 55: 217-50 (2003); Roberts, M. J., et al., Adv. Drug Deliv. Rev., 54: 459-76 (2002)), the contents of which are incorporated herein by reference.

In certain embodiments, specific mutations of antibodies can be made to alter the glycosylation of the polypeptide. Such mutations may be selected to introduce or eliminate one or more glycosylation sites, including but not limited to, O-linked or N-linked glycosylation sites. In certain embodiments, the proteins have glycosylation sites and patterns unaltered relative to the naturally-occurring proteins. In certain embodiments, a variant of proteins includes a glycosylation variant wherein the number and/or type of glycosylation sites have been altered relative to the naturally-occurring proteins. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created.

Monoclonal antibodies, and chimeric, and especially humanized antibodies, are of particular use for human therapeutic uses of the antibodies described herein. Monoclonal antibodies can be obtained by various techniques familiar to those skilled in the art. Briefly, spleen cells from an animal immunized with a desired antigen are immortalized, commonly by fusion with a myeloma cell (see, for example, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity ofa vertebrate host. Alternatively, one may isolate DNA sequences which encode a monoclonal antibody or a binding fragment thereof by screening a DNA library from human B cells according to the general protocol outlined by Huse et al., Science 246: 1275-1281 (1989).

Further, monoclonal antibodies can be collected and titered against an LRP6 polypeptide in an immunoassay, for example, a solid phase immunoassay with the ligand immobilized on a solid support. In some embodiments, monoclonal antibodies can bind with a K_{d} of at least about 0.1 mM, e.g., at least about 1 μM, e.g., at least about 0.1 M or better, e.g., 0.01 μM or lower.

The immunoglobulins, including binding fragments and other derivatives thereof, of the present disclosure may be produced readily by a variety of recombinant DNA techniques, including by expression in transfected cells (e.g. immortalized eukaryotic cells, such as myeloma or hybridoma cells) or in mice, rats, rabbits, or other vertebrate capable of producing antibodies by well-known methods. In one aspect, nucleic acid sequences encoding the Wnt agonist antibody or antigen-binding portion thereof are provided. The disclosure also describes vectors and mammalian host cells comprising the nucleic acid sequences. In some embodiments, the mammalian host cell is a CHO, CHO-K1, CHO-S, ExpiCHO, CHO-DG44, CHO-Pro minus, HEK293A, HEK293F cell. In some embodiments, the disclosure provides methods for producing the monoclonal antibody or antigen-binding portion thereof, comprising culturing the host cell under conditions to allow for production of the monoclonal antibody or antigen-binding portion thereof. Suitable source cells for the DNA sequences and host cells for immunoglobulin expression and secretion can be obtained from a number of sources, such as the American Type Culture Collection (Catalogue of Cell Lines and Hybridomas, Fifth edition (1985) Rockville, Md).

In some embodiments, the antibodies are antibody fragments such as Fab, F(ab′)₂, Fv or scFv. The antibody fragments can be generated using any means known in the art including, chemical digestion (e.g., papain or pepsin) and recombinant methods. Methods for isolating and preparing recombinant nucleic acids are known to those skilled in the art (see, Sambrook et al., Molecular Cloning, A Laboratory Manual (2d ed. 1989); Ausubel et al., Current Protocols in Molecular Biologv (1995)). The antibodies can b^{e} expressed in a variety of host cells, including E. coli, other bacterial hosts, yeast, and various higher eukaryotic cells such as the COS. CHO, and HeLa cells lines and myeloma cell lines.

Competitive binding assays can be used to identify antibodies that compete with an antibody described herein for specific binding to LRP6. Any of a number of competitive binding assays known in the art can be used to measure competition between two antibodies to the same antigen. Briefly, the ability of different antibodies to inhibit the binding of another antibody can be tested. For example, antibodies can be differentiated by the epitope to which they bind using a sandwich ELISA assay. This can be carried out by using a capture antibody to coat the surface of a well. A subsaturating concentration of tagged-antigen can then be added to the capture surface. This protein can be bound to the antibody through a specific antibody:epitope interaction. After washing, a second antibody, which has been covalently linked to a detectable moiety (e.g., HRP, with the labeled antibody being defined as the detection antibody) can be added to the ELISA. If this antibody recognizes the same epitope as the capture antibody it would be unable to bind to the target protein as that particular epitope would no longer be available for binding. If however this second antibody recognizes a different epitope on the target protein it would be able to bind and this binding can be detected by quantifying the level of activity (and hence antibody bound) using a relevant substrate. The background can be defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity. In some embodiments, a first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

An antibody described herein can comprise an Fc polypeptide. The Fc polypeptide can be a wild-type Fc polypeptide, e.g., a human IgG1 Fc polypeptide. In some embodiments, an Fc polypeptide in an antibody described herein can include amino acid substitutions that modulate effector function.

Iv. Methods for Promoting Tissue Regeneration

The antibodies (including antibody fragments) described herein agonize the Wnt/pi-catenin signaling pathway and promote tissue regeneration. Various conditions and diseases may result in degeneration or loss of tissue such as, for example, bone tissue, intestine tissue, liver tissue, or brain tissue. The Wnt agonist antibodies described herein may be used to regenerate such tissue by agonizing the Wnt/0-catenin signaling pathway in cells of that tissue.

In some embodiments, the Wnt agonist antibodies may be used to promote cell differentiation or tissue regeneration in vitro by adding the antibody or an antigen-binding portion thereof to a cell in vitro.

In other embodiments, the antibody or an antigen-binding portion thereof is administered ex vivo. In such embodiments, cells or a portion of tissue are removed from an individual's tissue that is in need of regeneration, and the antibody or an antigen-binding portion thereof is administered to the cells or tissue ex vivo. Then the treated cells or tissue are returned to the individual.

In other embodiments, the Wnt agonist antibodies may be used to restore tissue in an individual in need thereof. In certain embodiments, the individual may have a disease or condition involving degeneration or loss of tissue such as, for example, bone tissue, intestine tissue, liver tissue, or brain tissue. In some embodiments, the individual has a disease or condition in which insufficient Wnt signaling contributes to the disease or condition and/or its progression. In some embodiments, the individual has age-induced osteoporosis, drug induced bone loss, osteogenesis imperfecta, microgravity-induced bone loss, inflammatory bowel disease, Crohn's disease, ulcerative colitis, Celiac disease, rheumatoid arthritis, diabetes, chronic kidney disease, juvenile arthritis, dementia, Alzheimer's disease, stroke, cirrhosis, hepatitis, chronic alcoholism, severe alcoholic hepatitis, diabetic retinopathy, wet age-related macular degeneration (AMD), Fuchs' dystrophy, limbal stem cell deficiency, dry AMD, Sjögren's dry eye, short bowel syndrome, hearing loss and/or an autoimmune disease affecting one or more of the bone tissue, intestine tissue, liver tissue, or brain tissue (e.g., primary biliary cholangitis). In some embodiments, the antibody or an antigen-binding portion thereof is administered to the individual. In some embodiments, the pharmaceutical composition comprising the Wnt agonist antibody or antigen-binding portion thereof described herein is administered by intravenous injection, intraperitoneal injection, or subcutaneous injection. In other embodiments, any one of various other means of administration known in the art may be used.

In some embodiments, the antibody or an antigen-binding portion thereof may be used to treat an individual in need thereof in combination with other treatments to prevent tissue loss or to promote tissue regeneration. For example, in some aspects, the antibody or an antigen-binding portion thereof is used to treat a patient experiencing bone loss, in addition to bisphosphonates; calcitonin: hormone therapy; parathyroid hormone (PTH) analog; parathyroid hormone-related protein (PTHrp) analog; RANK ligand (RANKL) inhibitor; romosozumab; or a combination thereof.

### V. Pharmaceutical Compositions

The Wnt agonist antibodies for promoting tissue regeneration and for treating tissue loss (e.g., loss of bone tissue, intestinal tissue, liver tissue, or brain tissue) can be provided in a pharmaceutical composition. The pharmaceutical compositions may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present disclosure (see, e.g., Remington's Pharmaceutical Sciences, 17th ed., 1989).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. The formulations of compounds can be presented in unit-dose or multi-dose sealed containers, such as ampoules and vials. Solutions and suspensions can be prepared from sterile powders, granules, and tablets of the kind previously described. The modulators can also be administered as a part of prepared food or drug.

In certain embodiments, the pharmaceutical composition can be selected for parenteral delivery. The preparation of such pharmaceutically acceptable compositions is within the ability of one skilled in the art. In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments when parenteral administration is contemplated, a therapeutic composition can be in the form of a pyrogen-free, parenterally acceptable aqueous solution comprising a Wnt agonist antibody, in a pharmaceutically acceptable vehicle. In certain embodiments, a vehicle for parenteral injection is sterile distilled water in which the Wnt agonist antibody is formulated as a sterile, isotonic solution, and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

The dose administered to a patient should be sufficient to effect a beneficial response in the subject over time. The optimal dose level for any patient will depend on a variety of factors including the efficacy of the antibody employed, the age, body weight, physical activity, and diet of the patient, and on a possible combination with other drugs. The dose also will be determined by the existence, nature, and extent of any adverse side-effects that accompany the administration of a particular compound or vector in a particular subject.

In determining the effective amount of the agonist antibodies to be administered, a physician may evaluate circulating plasma levels of the agonist antibody and agonist antibody toxicity. In general, the dose equivalent of an agonist antibody is from about 1 ng/kg to 10 mg/kg for a typical subject. In some embodiments, the dose range for sub-cutaneous or iv administration is 0.1-20 mg/kg, e.g., 0.3-10 mg/kg.

For administration, the Wnt agonist antibodies can be administered at a rate determined by the EC₅₀ of the agonist, and the side-effects of the agonist at various concentrations, as applied to the mass and overall health of the subject. Administration can be accomplished via single or divided doses.

The compositions for treating or preventing tissue loss may be administered on a regular basis (e.g., weekly) for a period of time (e.g., 1, 2, 3, 4, 5, 6, 7, 8, 9, 10, 11, months or 1-3 years or more).

### VI. Methods for Identifying Wnt Agonist Antibodies

The present disclosure also provides methods for identifying a monoclonal antibody or an antigen-binding portion thereof that agonizes Wnt signaling and does not compete with a Wnt ligand or inhibitor. In some embodiments, the methods include: a) providing an LRP6 polypeptide or a portion thereof comprising at least the LRP6 polypeptide P3E3P4E4 domain; b) contacting the LRP6 polypeptide or portion thereof with a library of binding molecules; c) selecting one or more binding molecules from the library that bind to the LRP6 polypeptide or portion thereof; and d) identifying selected binding molecules that do not compete with a Wnt ligand or inhibitor for binding to the LRP6 polypeptide or portion thereof. In certain embodiments, the binding molecule is an antibody. In other embodiments, the binding molecule may be an aptamer, ligand, peptide or small molecule.

### EXAMPLES

The following examples are offered to illustrate, but not to limit the claimed invention.

### Example 1—Materials and Methods

### Cell Lines

Human embryonic kidney (HEK) 293A and 293, multiple myeloma H929, MM1.S, MM1.R, and RPMI8226 cell lines, mouse L Wnt-3a cell line, mouse pre-osteoblast MC3T3-E1 cell line, and mouse mesenchymal C3H/10T1/2 cell line were obtained from American Type Culture Collection (ATCC). Cells were maintained in DMEM, RPMI1640, or α-MEM supplemented with 10% FBS (Fisher Scientific) and 100 μg/ml penicillin/streptomycin (Axenia BioLogix) at 37° C. in a humidified atmosphere containing 5% CO2. Myeloma cell line-derived conditioned medium (CM) was collected by centrifugation of cell culture at 70˜80% confluency. All cell lines were used within six passages and were not authenticated by short tandem repeat profiling. Cells were tested negative (last test was performed in September 2020) for Mycoplasma using a PCR Mycoplasma detection kit (abm, Canada).

Selection of Wnt Agonist Antibodies from Phage Display Libraries

Recombinant LRP6 P3E3P4E4 domain was produced as a Fc fusion protein and purified on protein A column as previously described (Lee et al., 2018). Naïve phage antibody display libraries were selected on biotin-labeled LRP6-P3E3P4E4 fragment as previously described (Lee et al., 2018). After three rounds of selection, monoclonal phage were arrayed into 96-well plates and tested for binding to LRP6-transfected HEK293 cells by flow cytometry. Unique scFv antibodies were sequenced and identified from LRP6-binding phages, and individual phage clones were amplified and purified for further characterization.

### Plasmids, Cloning, and Site-Directed Mutagenesis

Full-length human LRP6 was cloned into pCMV-Entry (Origene) and utilized for sub-cloning, point mutation, or transfection. Truncate constructs of LRP6 ectodomains were cloned into pCMV-Entry and used for transient expression. Alanine mutants of LRP6 ectodomain were constructed using QuikChange Site-Directed Mutagenesis Kit (Agilent Technologies) according to the manufacturer's protocol. pFUSE-hIgG1-Fc2 (InvivoGen) was used for cloning of Fc-fusion constructs. Antibody genes were cloned into Abvec Ig-γ and Ig-κ plasmids kindly provided by Dr. Patrick Wilson at University of Chicago (Smith et al., Nature Protocols 4:372-84 (2009) with modifications (Lee et al., 2018). The TCF/LEF luciferase reporter SuperTopFlash (STF) and the control pRL-SV40 Renilla luciferase constructs (Addgene) were used for Wnt/β-catenin-responsive reporter assays. Wnt ligands were provided by transient co-transfection of pcDNA-Wnt1 or -Wnt3a expression plasmid (Addgene).

### Production of Recombinant Proteins and Antibodies

To construct Fc-fusions, LRP6-P3E3P4E4 or scFv genes were cloned into the pFUSE-hIgG1-Fc2 plasmid. To construct IgG, variable heavy (VH) and kappa light (Vκ) chain genes were sub-cloned into the original or modified Abvec, as described previously (Lee et al., 2018). For Fab constructs, CH2-CH3 was deleted from Ig-γ Abvec and a 6× His-tag was introduced at the C-termini of CH1. For transient transfection, plasmid DNA was resuspended in Opti-MEM (Life Technologies), mixed with polyethylenimine, and added to HEK293A cells. After 24 hours transfection, media was changed to Freestyle 293 expression medium (Gibco) and the cells were further cultured for 6-8 days. Secreted proteins in supernatants were collected, filtered, and purified on protein A agarose (Thermo Scientific) for Fc-fusions and IgGs or Ni-NTA resin (Thermo Scientific) for Fab according to the manufacturer's protocols.

### SuperTopFlash (STF) Luciferase Reporter Assays

Cells cultured in a 24- or 96-well plate were transiently transfected with STF luciferase reporter and pRL-SV40 plasmids using TransIT-2020 (Mirus Bio), with or without Wnt1- or Wnt3a-expression construct. To express LRP6 truncates and alanine mutants, plasmid DNA encoding the constructs was co-transfected with reporter plasmids into HEK293 cells. Antibodies diluted in culture medium were added on the transfected cells with recombinant DKK1 or RSPO2 (R&D Systems) and further incubated for 16 hours. Firefly luciferase (FL) and Renila luciferase (RL) activities were detected using Dual-Luciferase Reporter Assay System (Promega) and normalized as described previously (Lee et al., 2018). Data were expressed as a fold relative to a control group transfected only with reporter constructs.

### Apparent K_{D} Determination

The apparent K_{D} of antibodies was analyzed by FACS as described (Lee et al., 2018). Briefly, cells were trypsinized, washed, and resuspended in FACS buffer (PBS, 1% FBS). Antibodies serially diluted in FACS buffer were incubated with target cells (3×10⁵ cells/tube) overnight at 4° C. Cells were washed, incubated with Alexa Fluor® 647-labeled goat anti-human IgG (Jackson ImmunoResearch). 1 hour post incubation, cells were washed in PBS and analyzed using a BD Accuri C6 flow cytometer (BD Biosciences). Median Fluorescence Intensity (MFI) values were analyzed by curve fitting (GraphPad) to determine the affinity.

### Bio-Layer Interferometry

Competitive binding activity between anti-LRP6 Fab and recombinant DKK1 or Wnt3a to LRP6 ectodomain was estimated by bio-layer interferometry (BLI) using a BLitz (ForteBio) instrument. Protein A biosensors (ForteBio) were loaded with human LRP6-ECD-Fc (R&D Systems) for 120 seconds, and dipped in recombinant DKK1 or Wnt3a for 75 seconds followed by a mixture of DKK1+Wnt3a. DKK1+anti-LRP6 Fab, or Wnt3a+anti-LRP6 Fab for 75 seconds. Baselines were determined for 30 seconds before and after the loading step according to the manufacturer's instructions. For K_{D} measurement, Streptavidin biosensors (ForteBio) were loaded with biotinylated LRP6-P3E3P4E4-Fc for 120 seconds, and dipped in IgG or Fab for 120 seconds followed by dissociation within PBS for 120 seconds.

### Antibody-Receptor Docking Analysis

Antibody variable fragment (Fv) consisting of anti-LRP6 VH and Vκ sequences was generated by homology modeling using Rossetta Antibody (Weitzner et al., Nature Protocols 12:401-16 (2017). Docking models between the Fv and LRP6-P3E3 domain obtained from 3S8Z (Cheng et al., Nature Structural & Mol. Biol. 18:1204-U1244 (2011)) or 4A0P (Chen et al., 2011) were generated using ZDOCK (Pierce et al., Bioinformatics (2014)). Wnt3a- or Fv-binding residues in docking models were analyzed and visualized using the PyMOL Molecular Graphics System (Schrödinger, LLC).

### Osteogenic Differentiation

Differentiation of C3H10T1/2 cells was induced as described previously (Zhong et al., 1481:119-25 (2016)). In brief, C3H10T1/2 cells were cultured in normal growth medium (α-MEM, 10% FBS, 100 μg/ml penicillin/streptomycin) in 24-well culture plates at 70˜80% confluency. The following day, the culture medium was changed to osteogenic medium (growth medium supplemented with 50 μg/ml ascorbic acid, 10 mM β-glycerol phosphate) and replaced with osteogenic medium every 2-3 days. Cells were either only cultured in the osteogenic medium or treated with a combination of antibodies and/or 30% L cell Wnt3a-conditioned medium (Wnt3aCM) for 21 days. To determine matrix mineralization, cells were stained using Alizarin Red S (ARS) Staining Quantification Assay (ScienCell Research Laboratories), and images were taken using BIOREVO BZ-9000 microscope (Keyence). ARS dyes were extracted from the stained cells and quantified according to manufacturer's instructions.

Quantitative Real-Time PCR (qRT-PCR)

Osteogenic differentiation of C3H/10T1/2 cells was induced as described above for 3 days. Total RNA was isolated from the cells using TRIzol™ reagent (Invitrogen) and used to generate cDNA using High-Capacity cDNA Reverse Transcription Kit (Applied Biosystems) according to the manufacturer's protocol. qRT-PCR was performed with 15 ng of cDNA using Power SYBR Green PCR Master Mix (Applied Biosystems) on the ABI 7300 real time PCR system (Applied Biosystems). All reactions were conducted in duplicate and copy numbers for a target gene transcript were normalized to GAPDH. Data are presented as the relative mRNA expression in antibody-treated cells vs. control cells. Specific primer sets for each target gene were as follow:

GAPDH-F:
(SEQ ID NO: 29)
5′-GGCCTCACCCCATTTGATGT-′3,
GAPDH-R:
(SEQ ID NO: 30)
5′-CATGTTCCAGTATGACTCCACTC-′3,
ALP-F:
(SEQ ID NO: 31)
5′-AACCCAGACACAAGCATTCC-′3,
ALP-R:
(SEQ ID NO: 32)
5′-GCCTTTGAGGTTTTTGGTCA-′3,
RUNX2-F:
(SEQ ID NO: 33)
5′-GAATGGCAGCACGCTATTAAATCC-′3,
RUNX2-R:
(SEQ ID NO: 34)
5′-GCCGCTAGAATTCAAAACAGTTGG-′3,
BMP2-F:
(SEQ ID NO: 35)
5′-GGGACCCGCTGTCTTCTAGT-′3,
BMP2-R:
(SEQ ID NO: 36)
5′-TCAACTCAAATTCGCTGAGGAC-′3,
OC-F:
(SEQ ID NO: 37)
5′-CTGACCTCACAGATGCCAA-′3,
OC-R:
(SEQ ID NO: 38)
5′-GGTCTGATAGTCTGTCACAA-′3.

### Alkaline Phosphatase (ALP) Activity Assay

Cells were incubated with antibodies and Wnt3aCM (30%) for 7 days in the osteogenic medium as described above, washed, harvested, and lysed by repetitive freezing-thawing cycles in NP-40 buffer (150 mM NaCl, 1.0% NP-40, 50 mM Tris, pH 8.0) supplemented with protease inhibitors (Cell Signaling Technology). ALP activity in cell lysates was measured using p-nitrophenyl phosphate (Sigma-Aldrich) according to manufacturer's instructions. ALP activity was normalized against the control group without antibody and Wnt3a treatment.

### In Vivo Micro-CT Scanning

The micro x-ray computed tomography (micro-CT), a component of VECTor4/CT (MILabs B.V., Utrecht, The Netherlands) preclinical imaging system was used for the investigation. In order to visualize the femur and its joints, the field of view of micro-CT was set around the femur using built-in optical cameras, followed by CT acquisition with x-ray tube settings of 50 kVp and 0.24 mA. A total of 1,440 projections over 360° were acquired in a step-and-shoot mode with x-ray exposure time of 75 ms at each step. No data binning was applied during acquisition (i.e., 1×1 binning). During the CT data acquisition, animals were kept under anesthesia using isoflurane (approximately 2% isoflurane mixed with medical-grade oxygen). Image reconstruction after the projections were acquired was performed using the vendor-provided conebeam filtered backprojection algorithm. The reconstructed image volumes were in the voxel size of 0.02 mm×0.02 mm×0.02 mm. The volumetric matrix sizes were dependent on the field of view selected during the reconstruction step only focusing on distal femurs. After the reconstruction, the image volumes were processed to show the common orientation by re-orienting the isotropoic volumes using PMOD (PMOD Technologies, Zurich, Switzerland).

### In Vivo Bone Formation Study

2×10⁵ MM1.S cells were implanted in the right femur of NOD/SCID/IL-2Rγ^{-/-} (NSG) female mice. One week later, mice were randomized (n=5/group) and treated intraperitoneally with the vehicle (PBS) or the 66 IgG at 10 mg/kg every week for a total of 6 injections. One week post treatment, mice were anesthetized and scanned by micro-CT. One week post CT scanning, blood and femurs were collected from the mice, and free human Ig-lambda light chains in serum was assessed using a Human Lambda ELISA Kit (Bethyl Laboratories) according to manufacturer's instructions. All mouse studies were performed according to UCSF Institutional Animal Care and Use Committee-approved protocols.

### Trabecular and Cortical Bone Image Analysis

CT data files were utilized for 3D reconstruction or generating planar images of whole, distal, and proximal regions of the femurs using BoneJ2 plug-in operated by Fiji software as described previously (Doube et al., Bone 47:1076-79 (2010); Schindelin et al., Nature Methods 9:676-82 (2012)). The micro-architectural parameters of trabecular and cortical bones were analyzed using stacked 3D bone images, and bone volume over tissue volume (BV/TV), trabecular bone thickness (Tb.Th), and cortical thickness (Ct.Th) were obtained.

### Immunohistochemistry

Femurs were dissected to remove soft tissue, fixed in 10% neutral-buffered formalin, and decalcified in 14% EDTA for 4 weeks. The tissues were embedded in paraffin and cut into 4 mm sizes. Tissue sections were stained with hematoxylin and eosin (H&E) or anti-human Ig-lambda light chain antibody (Abcam) as described previously (Su et al., JCI Insight 3 (2018)). Images of stained sections were photographed using a BIOREVO BZ-9000 microscope (Keyence).

### Statistical Analysis

All statistical analysis to determine P values were conducted using two-tailed student's T-tests and P<0.05 was used to reject the null hypothesis. For multiple group comparisons, One-Way ANOVA was used using the Tukey's test.

### Example 2—Identification of a Novel Human Wnt Agonist Monoclonal Antibody

To identify novel LRP6-binding Wnt pathway agonist antibodies, a recombinant fragment of the extracellular domain of LRP6, specifically the P3E3P4E4 domain, was generated. Recombinant LPR6 P3E3P4E4 domain was produced as a Fc fusion protein and purified on protein A column as previously described (Lee et al., Scientiic Reports 8 (2018)). To construct Fc-fusions, LRP6-P3E3P4E4 or scFv genes were cloned into the pFUSE-hIgG1-Fc2 plasmid. To construct IgG, variable heavy (VH) and kappa light (Vκ) chain genes were sub-cloned into the original or modified Abvec, as described previously (Lee et al. (2018)). For Fab constructs, CH2-CH3 was deleted from Ig-γ Abvec and a 6× His-tag was introduced at the C-termini of CH1. For transient transfection, plasmid DNA was resuspended in Opti-MEM (Life Technologies), mixed with polyethylenimine, and added to HEK293A cells. After 24 hours transfection, media was changed to Freestyle 293 expression medium (Gibco) and the cells were further cultured for 6-8 days. Secreted proteins in supernatants were collected, filtered, and purified on protein A agarose (Thermo Scientific) for Fc-fusions and IgGs or Ni-NTA resin (Thermo Scientific) for Fab according to the manufacturer's protocols.

Naïve phage antibody display libraries were selected against this LRP6 fragment that had been biotin-labeled, and binding clones were identified. After three rounds of selection, monoclonal phage were arrayed into 96-well plates and tested for binding to LRP6-transfected HEK293 cells by flow cytometry. These LRP6-binding clones were tested for agonist effect on canonical Wnt signaling using the SuperTopFlash (STF) reporter assay. Cells cultured in a 24- or 96-well plate were transiently transfected with STF luciferase reporter, and pRL-SV40 plasmids using TransIT-2020 (Mirus Bio), with or without Wnt1- or Wnt3a-expression construct. To express LRP6 truncates and alanine mutants, plasmid DNA encoding the constructs was co-transfected with reporter plasmids into HEK293 cells. Antibodies diluted in culture medium were added on the transfected cells with recombinant DKK1 or RSPO2 (R&D Systems) and further incubated for 16 hours. Firefly luciferase (FL) and Renila luciferase (RL) activities were detected using Dual-Luciferase Reporter Assay System (Promega) and normalized as described previously (Lee et al. (2018)). Data were expressed as a fold relative to a control group transfected only with reporter constructs.

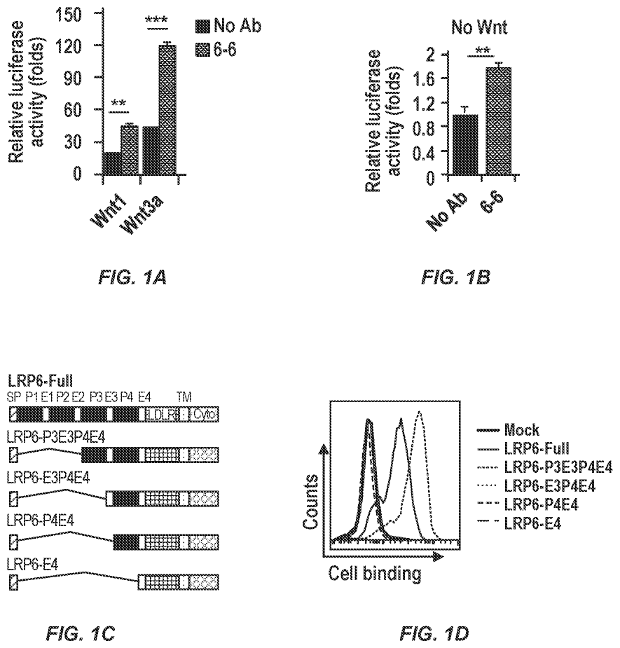

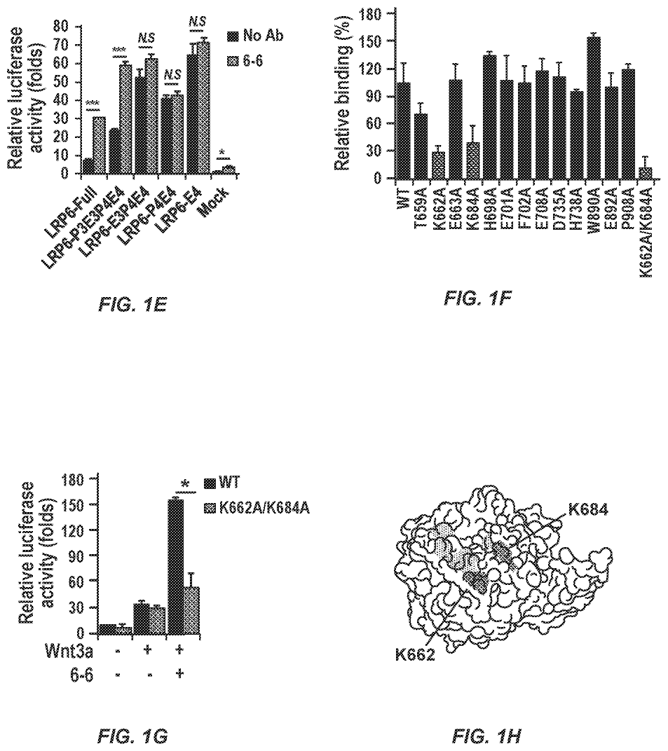

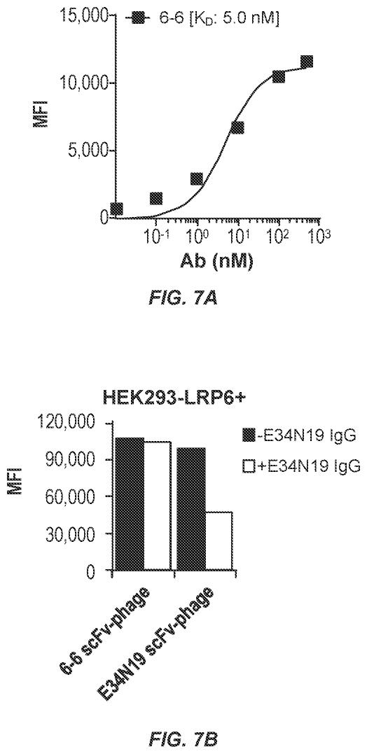

Unique scFv antibodies were sequenced and identified from LRP6-binding phages, and individual phage clones were amplified and purified for further characterization. One antibody, 66, was identified to have agonist activity initially as a phage-displayed scFv and then IgG (FIG. 1A). HEK293 cells transfected with STF reporter and different Wnt ligand expression constructs were incubated with or without the 66 IgG (100 nM). Error bars represent SD for n=2. **P<0.01, ***P<0.001. Wnt-agonist activity of the 66 IgG in the absence of endogenous Wnt ligands was tested. HEK293 cells transfected with the STF reporter constructs were incubated with or without 66 IgG (100 nM), and luciferase activity was normalized against a control without antibody treatment. Interestingly, the 66 IgG showed induction of Wnt/f-catenin signaling even in the absence of exogenously added Wnt ligands (FIG. 1B), indicating that this antibody possesses Wnt ligand-like properties. The apparent affinity of 66 IgG for LRP6 was measured on HEK293 cells and found to be ˜5 nM (FIG. 7A).

This novel canonical Wnt pathway agonist 66 antibody does not compete with a previously identified LRP6 P3E3 binder (FIG. 7B) that is antagonistic to Wnt/0-catenin signaling (Lee et al., 2018). HEK293 cells were transfected with LRP6 expression plasmid and incubated with the 66 IgG or E34N19 scFv-phage for 1 hour. E34N19 was identified previously as a Wnt antagonist binding to the P3E3P4E4 domain (Lee et al., 2018). E34N19 IgG was simultaneously added as a competitor. Bound phages were detected by sequential incubation of mouse anti-fd IgG and PE-labeled anti-mouse IgG. The results show that the agonist 66 IgG binds to a different site of LRP6.

### Example 3—a Novel Mechanism of Action: The 66 Agonist Antibody does not Operate as a Ligand Surrogate

To map where the 66 antibody binds to LRP6, a series of truncation mutants of the LRP6 P3E3P4E4 domain were generated (FIG. 1C; SP: Signal peptide; P3 and P4: Beta-propeller domain 3 and 4, respectively; E3 and E4: EGF-like domain 3 and 4, respectively; LDLR: Low-density lipoprotein receptor type A domain: TM: Transmembrane domain; Cyto: cytoplasmic domain). Full-length human LRP6 was cloned into pCMV-Entry (Origene) and utilized for sub-cloning, point mutation, or transfection. Truncate constructs of LRP6 ectodomains were cloned into pCMV-Entry and used for transient expression in HEK293 cells. The binding of the truncates to the 66 antibody was analyzed by flow cytometry (FIG. 1D). The 66 antibody was found to bind to the P3 domain of LRP6. The 66 agonist activity on LRP6 truncation mutants was further studied using the SIT reporter assay (FIG. 1E). The 66 IgG activated canonical Wnt signaling in cells expressing the LRP6 full-length and LRP6 P3E3P4E4 constructs but not other variants where the P3 domain is deleted, consistent with results from the cell binding study showing that the 66 antibody binds to the P3 domain.

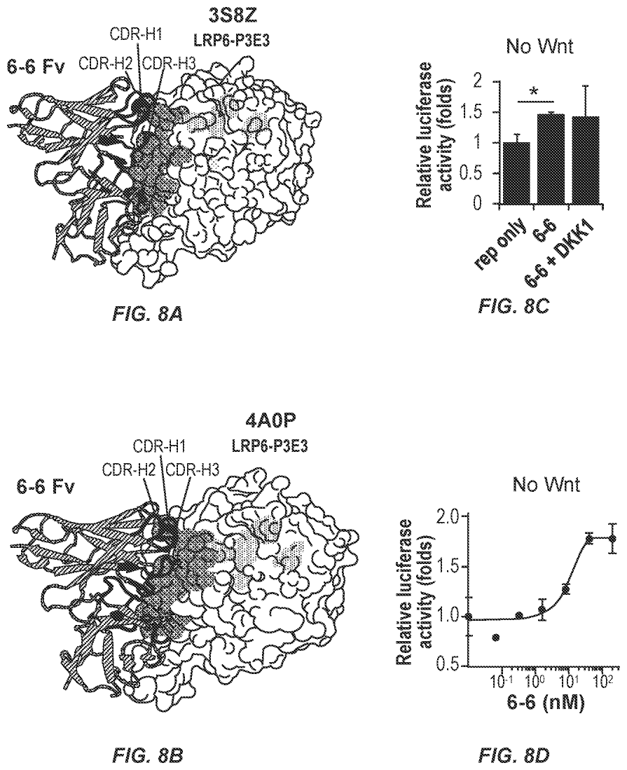

To map the binding site further, the binding of the 66 antibody to LRP6 was modeled using a homology modeling-predicted structure for the 66 Fv and two known crystal structures of LRP6 (3S8Z and 4A0P: FIGS. 8A and 8B, respectively). Several potential 66 contact sites were identified on LRP6. Alanine scan mutagenesis was performed at those sites. Alanine mutants of LRP6 ectodomain were constructed using QuikChange Site-Directed Mutagenesis Kit (Agilent Technologies) according to the manufacturer's protocol. Binding of the 66 IgG to these LRP6 mutants was analyzed by flow cytometry. K662A and K684A single mutations caused a significant loss of 66 binding (FIG. 1F). A double mutant (K662A/K684A) was produced and tested, and this double mutant exhibited a nearly complete loss of binding to the 66 antibody (FIG. 1F), thus confirming that K662 and K684 are critical contact sites. Consistent with the binding results, the 66-induced Wnt signaling activity was significantly decreased in HEK293 cells transfected with the double mutant (K662A/K684A) compared to the wild-type (WT) control (FIG. 1G). These two sites (red colored residues) are spatially distinct from known Wnt3a-binding sites (yellow colored residues, FIG. 1H), suggesting that the 66 antibody does not compete with ligand binding to LRP6.

The binding characteristics of the 66 antibody were further analyzed using biolayer interferometry. Competitive binding activity between anti-LRP6 Fab and recombinant DKK1 or Wnt3a to LRP6 ectodomain was estimated by bio-layer interferometry (BLI) using a BLitz (ForteBio) instrument. Protein A biosensors (ForteBio) were loaded with human LRP6-ECD-Fc (R&D Systems) for 120 seconds, and dipped in recombinant DKK1 or Wnt3a for 75 seconds followed by a mixture of DKK1+Wnt3a, DKK1+anti-LRP6 Fab (66 Fab), or Wnt3a+anti-LRP6 Fab for 75 seconds. Baselines were determined for 30 seconds before and after the loading step according to the manufacturer's instructions.

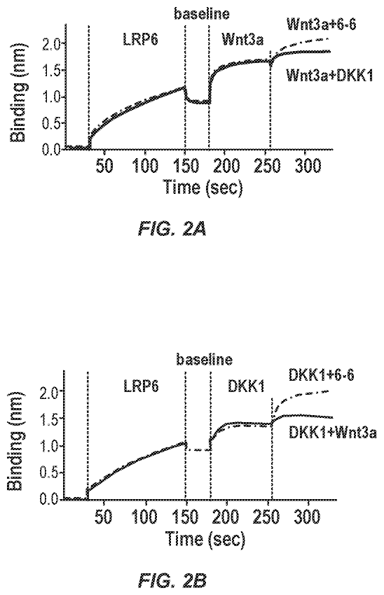

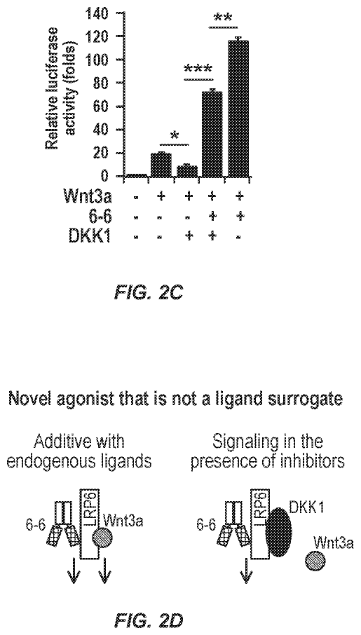

As expected by the epitope mapping result, the 66 Fab simultaneously bound to the LRP6-Wnt3a complex, whereas the mixture of Wnt3a and DKK1 could not additionally bind to the complex (FIG. 2A). This additive binding of 66 Fab was also observed when it was added to the LRP6-DKK1 complex (FIG. 2B), confirming that the 66 antibody does not compete with Wnt3a or DKK1 for LRP6 binding. This non-competitive binding of the 66 antibody was further studied by STF reporter assays. Even in the presence of the inhibitor DKK1, the 66 IgG was still able to significantly enhance signaling activity (FIG. 2C). These results suggest that the 66 antibody does not operate as a ligand surrogate. Instead, it works in parallel to Wnt ligands and is not subject to inhibition by endogenous Wnt inhibitors (FIG. 2D).

### Example 4—Amplification of Canonical Wnt Pathway Activation

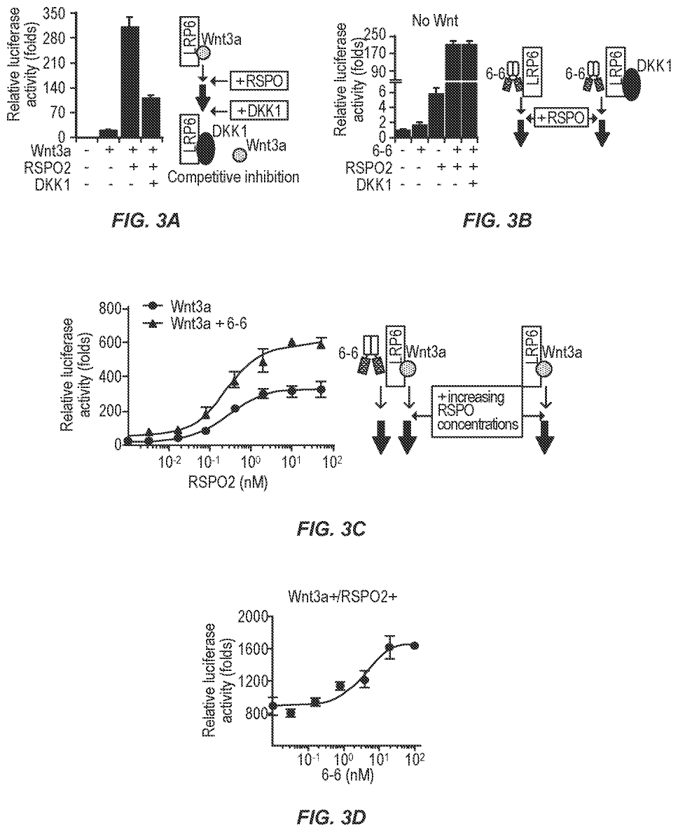

Since the 66 antibody does not bind to known Wnt ligand binding sites, the agonist effect of the 66 antibody was studied to determine whether the effect could be amplified by the Wnt signaling enhancer R-spondins (RSPOs). As a control, it was first shown that Wnt3a-induced Wnt/β-catenin signaling is greatly enhanced in the presence of RSPO2 and DKK1 addition significantly reduces the signaling enhancement (FIG. 3A). Next, the 66 antibody was studied in the context of RSPO2 in a similar manner. Interestingly, in the absence of exogenously provided Wnt ligands (either Wnt3a or Wnt1), the agonist effect of the 66 IgG can be dramatically elevated by addition of RSPO2, and this signaling enhancement was not affected by addition of DKK1 (FIG. 3B). Thus the 66 antibody acts like a new type of Wnt ligand whose agonist activity is independent from known Wnt ligands and amplified by R-spondin. Unlike known Wnt ligands, the 66 agonist activity is not inhibited by endogenous inhibitors.

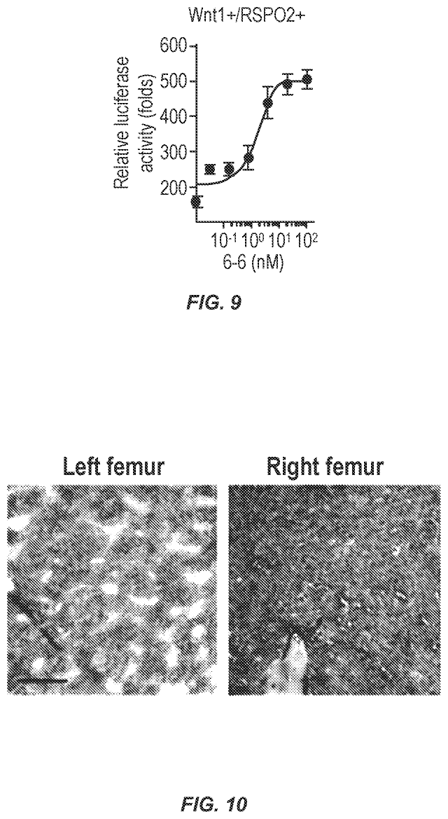

Next, the effect of increasing RSPO2 concentrations on the agonist activity of the 66 antibody in the presence of known Wnt ligands was evaluated. As shown in FIG. 3C, providing the 66 antibody (20 nM) increased the maximum Wnt ligand signaling activity amplified by RSPO2 (528-fold with 66 vs. 308-fold without 66). This effect was further studied over a range of 66 concentrations. In the presence of a constant concentration of the Wnt ligands (Wnt3a for FIG. 3D and Wnt1 for FIG. 9) and RSPO2, the 66 antibody showed dose-dependent agonist activities with EC50 of 4.8 nM and 1.8 nM for Wnt3a- and Wnt1-mediated β-catenin signaling, respectively. Thus, these data further support that the 66 antibody behaves like a new type of Wnt ligand that acts additively but not competitively with known Wnt ligand and responds to RSPO2-mediated signaling enhancement with or without known Wnt ligands.

### Example 5—the Wnt Agonist Antibody Induces Osteoblast Diffentiation

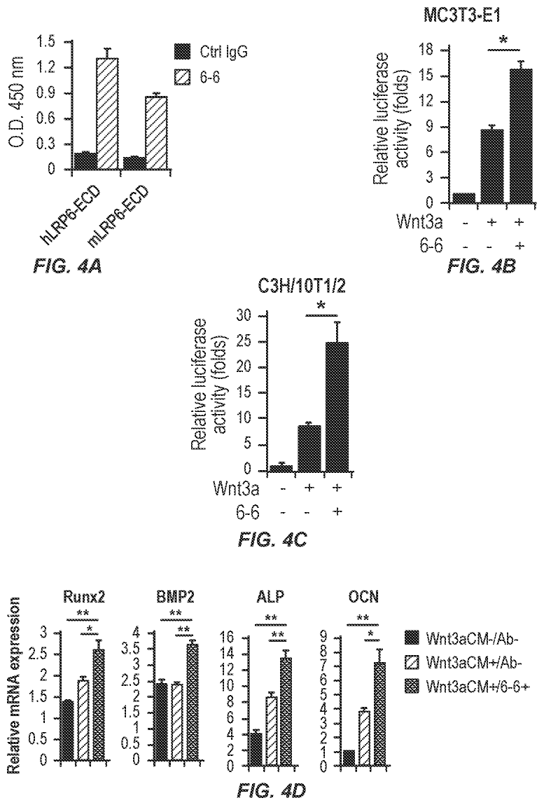

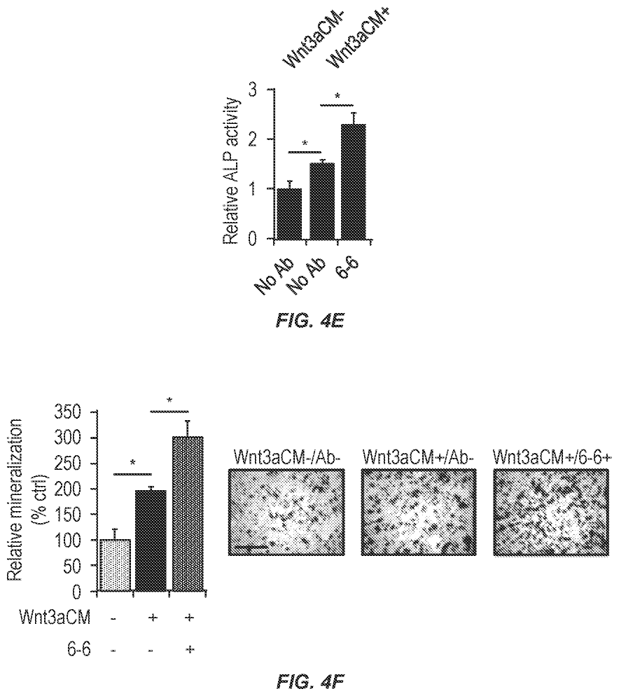

One of the biological consequences of canonical Wnt signaling activation is the induction of osteoblast differentiation and bone formation. The Wnt-agonist effect of the 66 IgG on mouse pre-osteoblast MC3T3-E1 and bone marrow-derived mesenchymal C3H/10T1/2 cell lines was analyzed. First, the cross-reactive binding of the 66 IgG to human and mouse LRP6 was analyzed. The 66 IgG specifically bound to both human and mouse LRP6 ectodomains (FIG. 4A). Then the effect of the 66 IgG on Wnt/β-catenin signaling activation by STF reporter assays was analyzed using the aforementioned mouse-derived cell lines. Wnt3a induced reporter activity, and addition of the 66 IgG significantly enhanced signaling in both MC3T3-E1 (FIG. 4B) and CH3/10T1/2 (FIG. 4C) cell lines.

Next, the induction of osteoblastic differentiation of C3H/10T1/2 cells was studied by measuring expression of osteoblast marker genes (RUNX2, BMP2, ALP, and OCN) by qRT-PCR. Osteogenic differentiation of C3H/10T1/2 cells was induced as described above for 3 days. Total RNA was isolated from the cells using TRIzol™ reagent (Invitrogen) and used to generate cDNA using High-Capacity cDNA Reverse Transcription Kit (Applied Biosystems) according to the manufacturer's protocol. qRT-PCR was performed with 15 ng of cDNA using Power SYBR Green PCR Master Mix (Applied Biosystems) on the ABI 7300 real time PCR system (Applied Biosystems). All reactions were conducted in duplicate and copy numbers for a target gene transcript were normalized to GAPDH. Data are presented as the relative mRNA expression in antibody-treated cells vs. control cells.

Specific primer sets for each target gene were as follows:

GAPDH-F:
(SEQ ID NO: 29)
5′-GGCCTCACCCCATTTGATGT-′3,
GAPDH-R:
(SEQ ID NO: 30)
5′-CATGTTCCAGTATGACTCCACTC-′3,
ALP-F:
(SEQ ID NO: 31)
5′-AACCCAGACACAAGCATTCC-′3,
ALP-R:
(SEQ ID NO: 32)
5′-GCCTTTGAGGTTTTTGGTCA-′3,
RUNX2-F:
(SEQ ID NO: 33)
5′-GAATGGCAGCACGCTATTAAATCC-′3,
RUNX2-R:
(SEQ ID NO: 34)
5′-GCCGCTAGAATTCAAAACAGTTGG-′3,
BMP2-F:
(SEQ ID NO: 35)
5′-GGGACCCGCTGTCTTCTAGT-′3,
BMP2-R:
(SEQ ID NO: 36)
5′-TCAACTCAAATTCGCTGAGGAC-′3,
OC-F:
(SEQ ID NO: 37)
5′-CTGACCTCACAGATGCCAA-′3,
OC-R:
(SEQ ID NO: 38)
5′-GGTCTGATAGTCTGTCACAA-′3.

As shown in FIG. 4D, the Wnt-agonist 66 induced expression of Wnt downstream genes involved in osteoblastic differentiation, with 66 combined with Wnt3a conditioned media (Wnt3aCM) being more potent than Wnt3aCM alone. In addition to mRNA expression, we also measured alkaline phosphatase activity (ALP) in C3H/10T1/2 cells incubated with 66. As shown in FIG. 4E, Wnt3aCM upregulated ALP activity, and the addition of 66 further increased ALP activity in the presence of Wnt3aCM.

In order to directly assess the osteoblastic commitment of C3H/10T1/2 cells, in vitro mineralization assays (Gregory et al., Analyt. Biochem. 329:777-84 (2004)) in the presence of the 66 antibody and Wnt3aCM were conducted. As shown in FIG. 4F, Wnt3aCM increased mineralization that was further enhanced by the addition of 66. These data suggest that the Wnt-agonist antibody 66 promotes osteoblast differentiation and acts additively with natural Wnt ligands.

### Example 6—the Wnt Agonist Antibody Induces Bone Formation

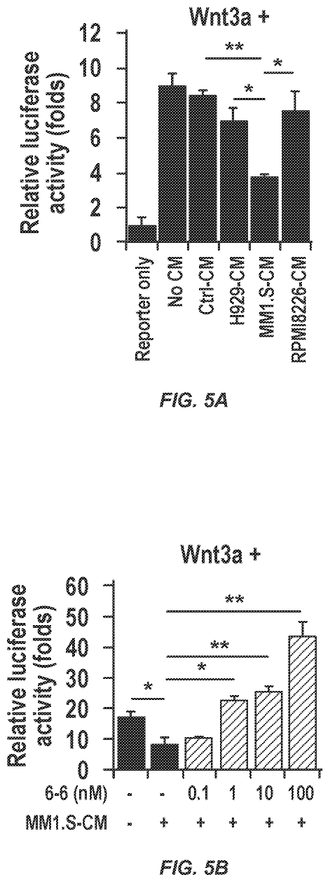

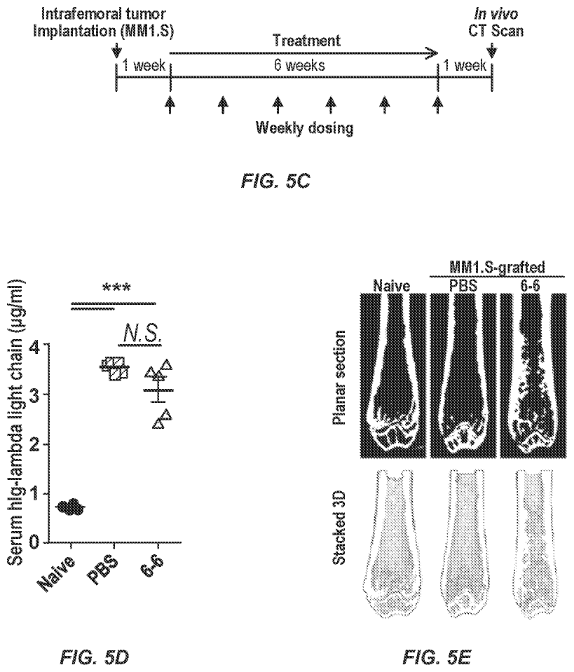

Certain types of primary and metastatic cancers locate to the bone and cause extensive bone remodeling in patients. Multiple myeloma resides in the bone marrow and is known to induce significant bone loss caused by the secretion of inhibitors of canonical Wnt signaling (Edwards, Blood 112:216-17 (2008). Glass el al., NEJM 349:2479-80 (2003)). Since the novel Wnt-agonist antibody 66 does not compete with known Wnt inhibitors for LRP6 binding, the 66 antibody could effectively counteract Wnt inhibitors produced by myeloma cells. To test this hypothesis, a panel of multiple myeloma cell lines were screened for the presence of Wnt inhibitors in conditioned media on HEK293 cells co-transfected with the STF reporter and Wnt3a-expression construct. As shown in FIG. 5A, conditioned media from MM1.S (MM1.S-CM) showed significant inhibitory effect compared to the control (no conditioned media) or other conditioned media from different multiple myeloma cell lines. Therefore, the MM1.S cells were selected for further study. Again using the HEK293 and STF reporter assay, in the presence of both Wnt3a and MM1 S-CM, the 66 IgG restored the signals inhibited by MM1.S-CM and even further stimulated signaling at higher concentrations of 66 treatment (FIG. 5B).

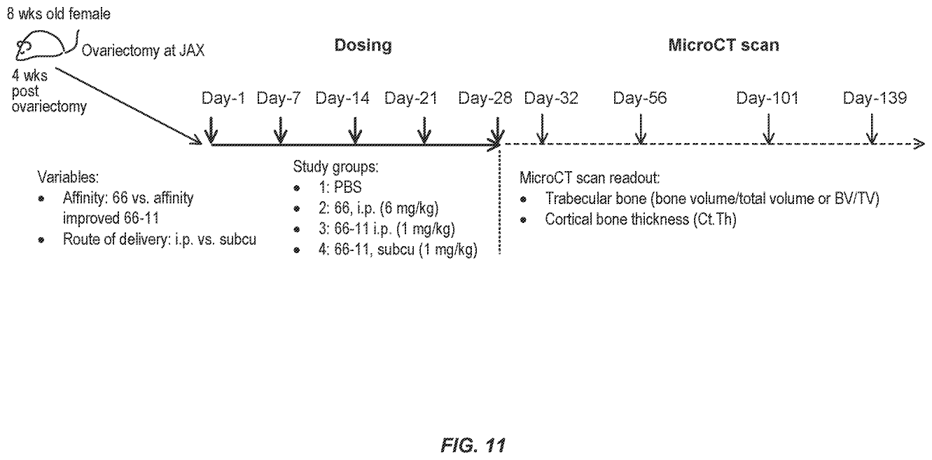

To address the effect of the 66 antibody on bone remodeling in vivo, an intrafemoral osteolytic model was established by implantation of MM1.S cells into the right femur of NSG mice. As outlined in FIG. 5C, antibody treatment started 1 week post implantation and continued for 6 weeks by weekly intraperitoneal (i.p.) injection. Live mice were scanned by micro-CT to assess changes of femoral bone structure. To confirm tumor establishment, we first analyzed human Ig-lambda light chain levels in serum by ELISA. As shown in FIG. 5D, the concentration of human Ig-lambda light chain in the MM1.S-implanted groups (PBS and 66) was significantly higher than that of the group with no MM1.S injection (Naive). The light chain levels in the 66 group were lower than those in the PBS group, but there was no statistically significant difference between the two groups (FIG. 5D). By immunohistochemistry study, MM1.S myeloma cells established in the right femur were also detected by anti-human Ig-lambda antibody (FIG. 10). The bone-forming effect of the 66 IgG was assessed by micro-CT analysis. Whole femurs were analyzed to generate planar and 3D images from CT-scanned data. As shown in FIG. 5E, MM1.S implantation resulted in osteolysis especially in the trabecular bone area (Naive vs. PBS). Strikingly, treatment with the 66 IgG reversed the femoral bone loss compared to the control (66 IgG vs. PBS), indicating that the Wnt-agonist antibody 66 promotes bone formation in vivo (FIG. 5E).

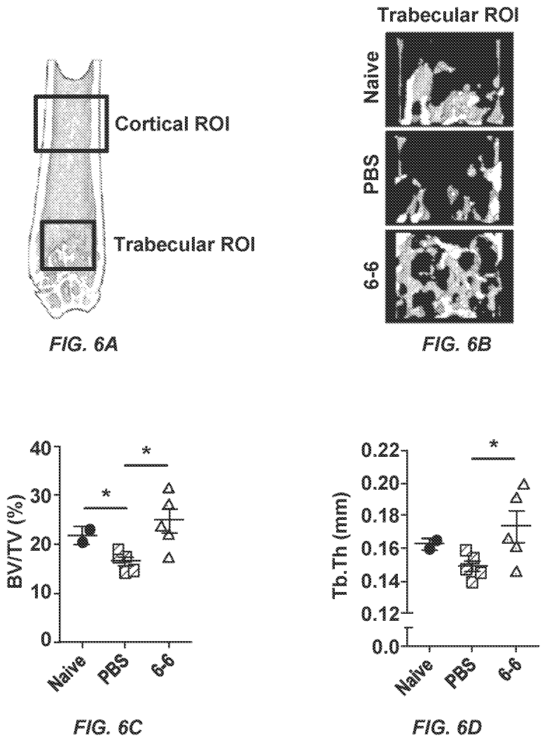

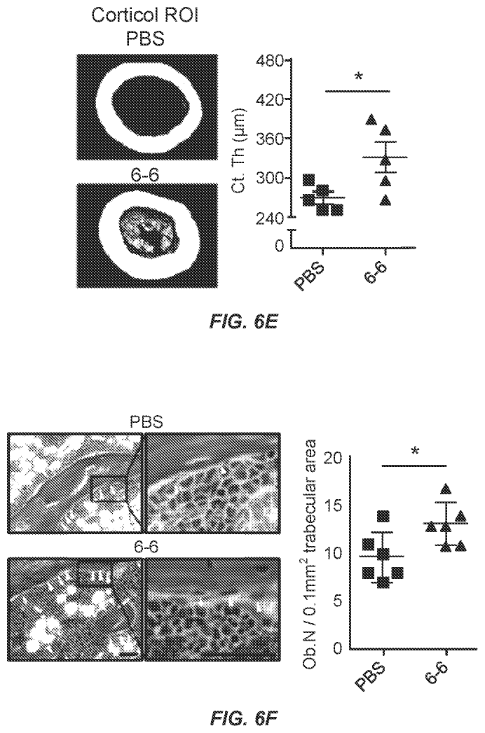

The quantitative bone formation effect of the 66 IgG was further investigated. Region of interest (ROT) to assess bone structures was designated as shown in FIG. 6A, and micro-CT images were reconstructed for 3D view and quantification of bone micro-architectures. By analyzing trabecular ROI, compared to the Naive group with no myeloma implantation, trabecular bone in the PBS group was catabolized by implanted MM1.S cells that secrete Wnt/β-catenin signaling inhibitors (FIG. 6B). However, consistent with the whole femur image analysis, 66 IgG treatment showed trabecular-anabolic activity (FIG. 6B). 66 IgG treatment resulted in a significant increase in bone volume over tissue volume (BV/TV) and trabecular bone thickness (Tb.Th) (FIGS. 6C and 6D, respectively). In addition to the distal femur, we analyzed cortical bone in the proximal femur legion. By reconstructing the cortical ROI and measuring cortical bone thickness (Ct.Th), we found that the 66 IgG significantly increased cortical bone formation compared to the control group (PBS) (FIG. 6E), suggesting that the 66 Wnt-agonist effect stimulates both trabecular and cortical bone formation. To assess 66 IgG effects on osteoblastic differentiation, the number of bone-lining osteoblasts was analyzed and found to be increased by 66 IgG treatment. Representative H&E staining images of the femurs is shown in FIG. 6F, which shows a significant increase in the number of osteoblasts on trabecular bone lining in the 66 treated group compared to the PBS group. Taken together, the novel Wnt-agonist antibody 66 reverses bone loss in the intrafemoral MM1 S myeloma model, demonstrating its potential in treating osteolytic diseases.

### Example 7—Wnt Agonist Antibodies Promote Bone Formation In Vivo in an Osteoporosis Mouse Model

Experiments were conducted to study the effect of Wnt agonist antibodies on bone tissue in an ovariectomy-induced osteoporosis mouse model (FIG. 11). The experiment evaluated two variables: (1) the antibody, and more specifically the affinity of the antibody (comparing the 66 antibody with its higher affinity variant 66-11 antibody (at ⅙ the dose of 66)); and (2) the route of delivery (comparing intraperitoneal (i.p.) injection with subcutaneous (subcu) injection). For readouts, both trabecular and cortical bone were evaluated.

Eight week old female C57BL/6j mice were ovariectomized by the Jackson Laboratory (JAX). Four weeks post-ovariectomy, the mice were treated with a Wnt agonist antibody (66 or 66-11) or a PBS control. The 66 antibody was administered intraperitoneally at 6 mg/kg, and the 66-1l antibody was administered intraperitoneally or subcutaneously at 1 mg/kg. For each study group, 6 mice were treated and analyzed. After 5 weekly doses, the mice were scanned by in vivo micro-CT (focusing on the distal femur bone) at the indicated time points (4 days, 28 days, 73 days, and 111 days after the final dose). For each mouse, both legs were scanned to generate images used for analysis by ImageJ (BoneJ).

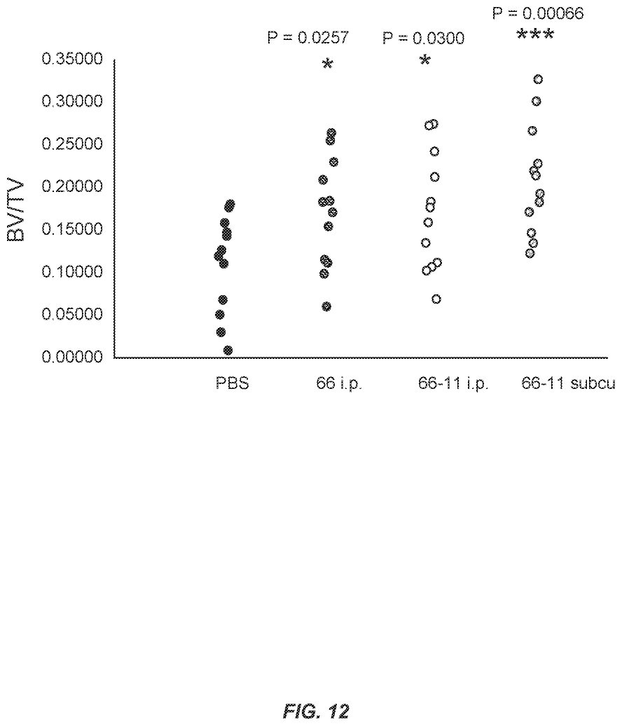

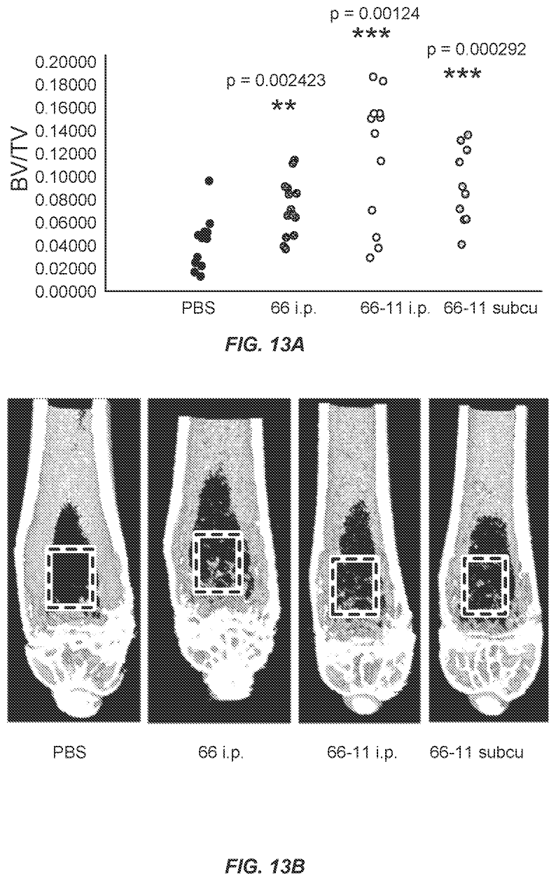

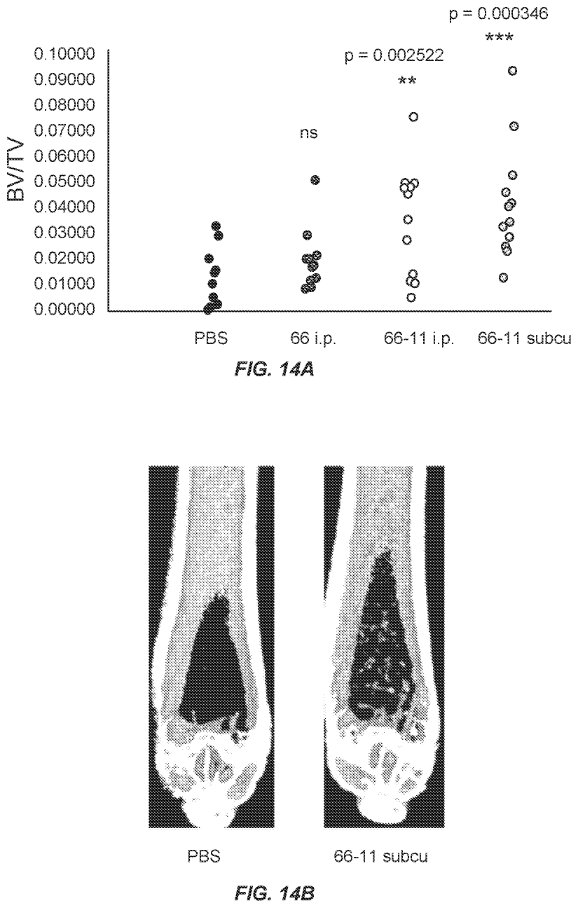

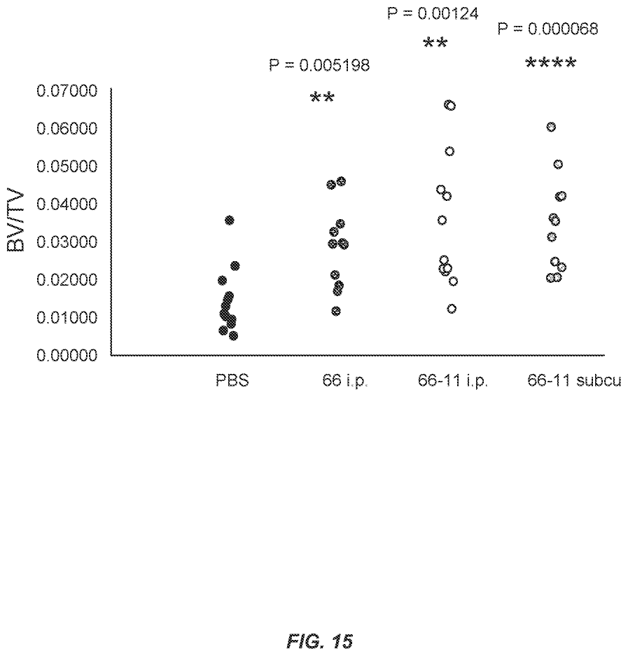

The results of the analysis of trabecular bone volume are shown in FIG. 12 (4 days after last dose), FIGS. 13A and 13B (28 days after last dose), FIGS. 14A and 14B (73 days after last dose), and FIG. 15 (111 days after last dose). Compared with the PBS control, the 66 antibody showed significant bone promoting activity at the first 2 time points analyzed. The 66-11 antibody showed significant bone promoting activity at all time points analyzed. Both the intraperitoneal and subcutaneous routes of delivery resulted in significant bone promoting activity.

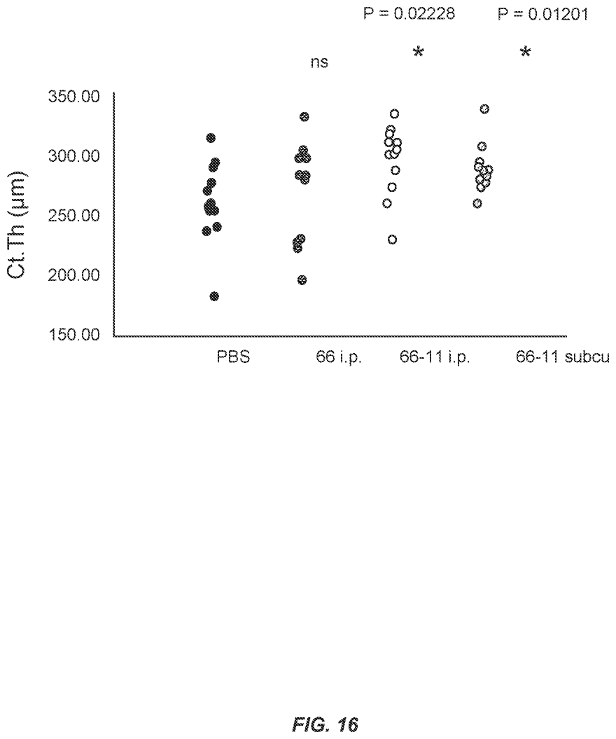

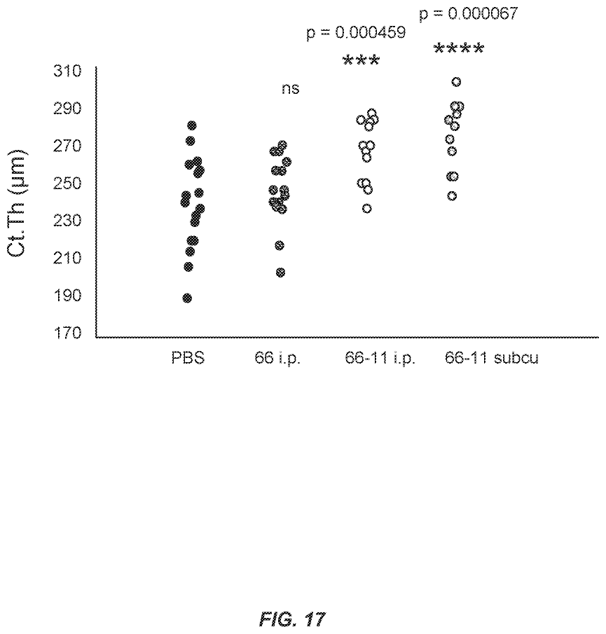

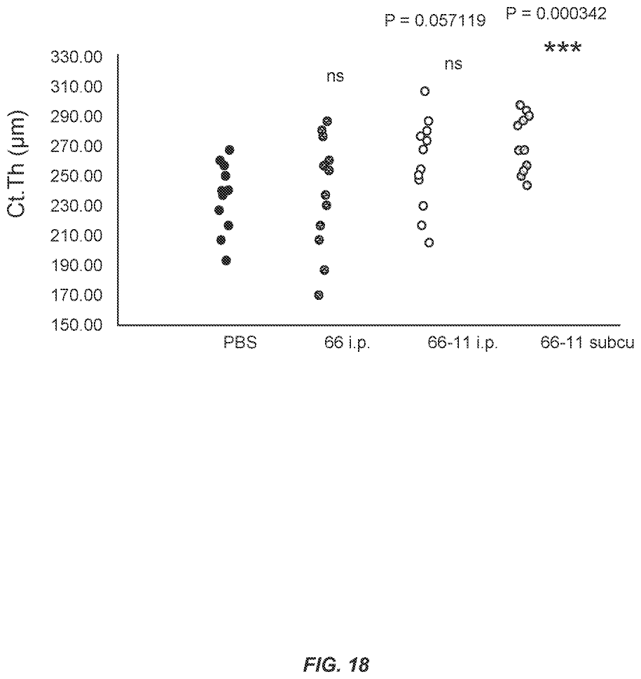

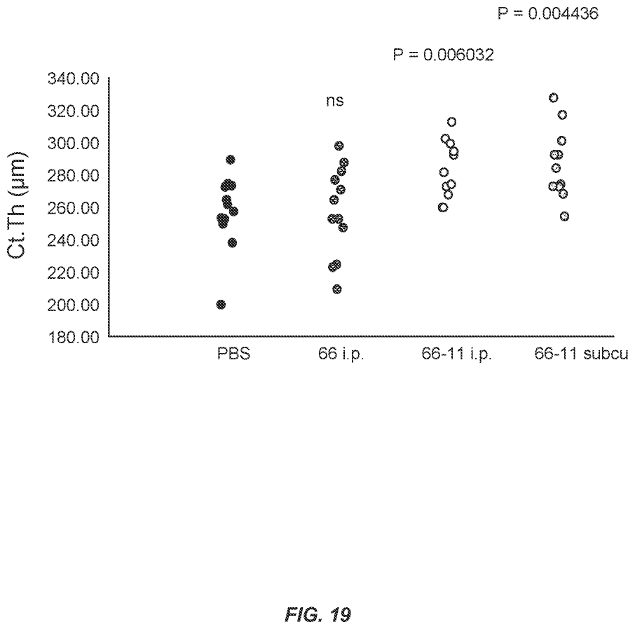

The results of the analysis of cortical bone thickness are shown in FIG. 16 (4 days after last dose), FIG. 17 (28 days after last dose), FIG. 18 (73 days after last dose), and FIG. 19 (111 days after last dose). Compared with the PBS control, the 66-11 antibody showed significant bone promoting activity at 3 of the time points when administered intraperitoneally or at all time points when administered subcutaneously.

These data show that the Wnt agonist antibodies are highly active in promoting bone formation in vivo in ovariectomized mice, with the higher affinity antibody 66-11 showing more potent activity even at ⅙th the dose of the parental antibody 66. Both intraperitoneal and subcutaneous routes of delivery resulted in potent bone promoting activity, and the effect is long lasting. No body weight loss or any overt toxicity was observed for the duration of the experiment (139 days).

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

1. A monoclonal antibody, or an antigen-binding portion thereof, that specifically binds to low density lipoprotein receptor-related protein 6 (LRP6) and agonizes Wnt signaling, wherein the antibody or antigen-binding portion comprises:
a) a heavy chain variable domain (VH) comprising
(1) the amino acid sequence of SEQ ID NO:2;
(2) the amino acid sequence of SEQ ID NO:8;
(3) heavy chain CDR1-3 comprising the amino acid sequences of SEQ ID NOs: 18, 19, and 21, respectively;
(4) heavy chain CDR1-3 comprising the amino acid sequences of SEQ ID NOs: 18, 19, and 22, respectively;
(5) heavy chain CDR1-3 comprising the amino acid sequences of SEQ ID NOs: 18, 19, and 23, respectively;
(6) heavy chain CDR1-3 comprising the amino acid sequences of SEQ ID NOs: 18, 19, and 24, respectively; or
(7) heavy chain CDR1-3 comprising the amino acid sequences of SEQ ID NOs: 18, 19, and 25, respectively; and
b) a light chain variable domain (VL) comprising
(1) the amino acid sequence of SEQ ID NO:15;
(2) light chain CDR1-3 comprising the amino acid sequences of SEQ ID NOs: 26, 27, and 29, respectively; or
(3) light chain CDR1-3 comprising the amino acid sequences of SEQ ID NOs: 26, 27, and 30, respectively.

2. The monoclonal antibody or antigen-binding portion thereof of claim 1, wherein the antibody comprises a VH comprising the amino acid sequence of SEQ ID NO:3 and a VL comprising the amino acid sequence of SEQ ID NO:15.

3. The monoclonal antibody or antigen-binding portion thereof of claim 1, wherein the antibody comprises a VH comprising the amino acid sequence of SEQ ID NO:3 and a VL comprising the amino acid sequence of SEQ ID NO:16.

4. The monoclonal antibody or antigen-binding portion thereof of claim 1, wherein the antibody comprises a VH comprising the amino acid sequence of SEQ ID NO:9 and a VL comprising the amino acid sequence of SEQ ID NO:16.

5. The monoclonal antibody or antigen-binding portion thereof of claim 1, wherein the antibody comprises a VH comprising the amino acid sequence of SEQ ID NO:2 and a VL comprising the amino acid sequence of SEQ ID NO:15.

6. The monoclonal antibody or antigen-binding portion thereof of claim 1, wherein the antibody comprises
a) a heavy chain comprising
(1) a heavy chain comprising HCDR1-3 comprising the amino acid sequences of SEQ ID NOs: 18, 19, and 21, respectively;
(2) heavy chain CDR1-3 comprising the amino acid sequences of SEQ ID NOs: 18, 19, and 22, respectively;
(3) heavy chain CDR1-3 comprising the amino acid sequences of SEQ ID NOs: 18, 19, and 23, respectively;
(4) heavy chain CDR1-3 comprising the amino acid sequences of SEQ ID NOs: 18, 19, and 24, respectively; or
(5) heavy chain CDR1-3 comprising the amino acid sequences of SEQ ID NOs: 18, 19, and 25, respectively; and
b) a light chain comprising the amino acid sequence of SEQ ID NO:15.

7. The monoclonal antibody or antigen-binding portion thereof of claim 6, wherein the antibody comprises a VH comprising the amino acid sequence of SEQ ID NO:3, 4, 5, 6, or 7, and a VL comprising the amino acid sequence of SEQ ID NO:15.

8. The monoclonal antibody or antigen-binding portion thereof of claim 1, wherein the antibody comprises
a) a heavy chain comprising
(1) the amino acid sequence of SEQ ID NO:8;
(2) heavy chain CDR1-3 comprising the amino acid sequences of SEQ ID NOs: 18, 19, and 21, respectively;
(3) heavy chain CDR1-3 comprising the amino acid sequences of SEQ ID NOs: 18, 19, and 22, respectively;
(4) heavy chain CDR1-3 comprising the amino acid sequences of SEQ ID NOs: 18, 19, and 23, respectively;
(5) heavy chain CDR1-3 comprising the amino acid sequences of SEQ ID NOs: 18, 19, and 24, respectively; or
(6) heavy chain CDR1-3 comprising the amino acid sequences of SEQ ID NOs: 18, 19, and 25, respectively; and
b) a light chain comprising
(1) light chain CDR1-3 comprising the amino acid sequences of SEQ ID NOs: 26, 27, and 29, respectively; or
(2) light chain CDR1-3 comprising the amino acid sequences of SEQ ID NOs:
26, 27, and 30, respectively.

9. The monoclonal antibody or antigen-binding portion thereof of claim 8, wherein the antibody comprises a VH comprising the amino acid sequence of SEQ ID NO:8, 9, 10, 11, 12, or 13, and a VL comprising the amino acid sequence of SEQ ID NO:16 or 17.

10. The monoclonal antibody or antigen-binding portion thereof of claim 1, wherein the antibody comprises a human IgG heavy chain constant region.

11. The monoclonal antibody or antigen-binding portion thereof of claim 10, wherein the antibody is an effector-attenuated IgG1 antibody.

12. The monoclonal antibody or antigen-binding portion thereof of claim 11, wherein the antibody is an IgG1 antibody comprising a leucine to alanine substitution at positions 234 and 235.

13. The monoclonal antibody or antigen-binding portion thereof of claim 10, wherein the antibody is an IgG2 antibody.

14. The monoclonal antibody or antigen-binding portion thereof of claim 1, wherein the antibody is human.

15. A monoclonal antibody or an antigen-binding portion thereof that agonizes Wnt signaling and does not compete with a Wnt ligand or inhibitor.

16. A pharmaceutical composition comprising the monoclonal antibody or antigen-binding portion thereof of claim 1 and a pharmaceutically acceptable excipient.

17. A nucleic acid sequence encoding the monoclonal antibody or antigen-binding portion thereof of claim 1.

18. A vector comprising the nucleic acid sequence of claim 17.

19. A mammalian host cell comprising the nucleic acid sequence of claim 17.

20. A method for producing a monoclonal antibody or antigen-binding portion thereof, the method comprising culturing the host cell of claim 19 under conditions to allow for production of the monoclonal antibody or antigen-binding portion thereof.

21. A method for promoting tissue regeneration, the method comprising
adding the monoclonal antibody or antigen-binding portion thereof of claim 1 to a cell in vitro or ex vivo.

22. A method for restoring tissue in an individual in need thereof, comprising administering to the individual the pharmaceutical composition of claim 16.

23. The method of claim 21, wherein the tissue is bone tissue, intestine tissue, liver tissue, or brain tissue.

24. The method of claim 21, wherein the individual has a disease or condition characterized by insufficient Wnt signaling.

25. The method of claim 21, wherein the individual has a disease or condition selected from the group consisting of age-induced osteoporosis, drug-induced bone loss, osteogenesis imperfecta, inflammatory bowel disease, severe alcoholic hepatitis, diabetic retinopathy, wet age-related macular degeneration (AMD), Fuchs' dystrophy, limbal stem cell deficiency, dry AMD, Sjögren's dry eye, short bowel syndrome, and hearing loss.

26. A method for identifying a monoclonal antibody or an antigen-binding portion thereof that agonizes Wnt signaling and does not compete with a Wnt ligand or inhibitor, comprising:
a) providing an LRP6 polypeptide or a portion thereof comprising at least the LRP6 polypeptide P3E3P4E4 domain;
b) contacting the LRP6 polypeptide or portion thereof with a library of binding molecules;
c) selecting one or more binding molecules from the library that bind to the LRP6 polypeptide or portion thereof; and
d) identifying selected binding molecules that do not compete with a Wnt ligand or inhibitor for binding to the LRP6 polypeptide or portion thereof.


SEQUENCE LISTING (40 sequences)

SEQ ID NO: 1 - protein, 123 aa, Synthetic construct
QVQLLQSGGGLVQPGGSLRLSCAASGFTFSTYAMSWVRQAPGKGLEWVSTIGPSGSSTYYAYADSVKGRFTISRDNYKNMLYLQMNSLRAEDTAVYYCAKEGPNSGYFDFDYWGQGTLVTVSS

SEQ ID NO: 2 - protein, 123 aa, Synthetic construct
EVQLLESGGGLVQPGGSLRLSCAASGFTFSTYAMSWVRQAPGKGLEWVSTIGPSGSSTYYAYADSVKGRFTISRDNSKNTLYLQMNSLRAEDTAVYYCAKEGPNSGYFDFDYWGQGTLVTVSS

SEQ ID NO: 3 - protein, 123 aa, Synthetic construct
EVQLLESGGGLVQPGGSLRLSCAASGFTFSTYAMSWVRQAPGKGLEWVSTIGPSGSSTYYAYADSVKGRFTISRDNSKNTLYLQMNSLRAEDTAVYYCAKESPFSAYFTFDYWGQGTLVTVSS

SEQ ID NO: 4 - protein, 123 aa, Synthetic construct
EVQLLESGGGLVQPGGSLRLSCAASGFTFSTYAMSWVRQAPGKGLEWVSTIGPSGSSTYYAYADSVKGRFTISRDNSKNTLYLQMNSLRAEDTAVYYCAKEGPNSGYFTFDYWGQGTLVTVSS

SEQ ID NO: 5 - protein, 123 aa, Synthetic construct
EVQLLESGGGLVQPGGSLRLSCAASGFTFSTYAMSWVRQAPGKGLEWVSTIGPSGSSTYYAYADSVKGRFTISRDNSKNTLYLQMNSLRAEDTAVYYCAKEGPFSGYFTFDYWGQGTLVTVSS

SEQ ID NO: 6 - protein, 123 aa, Synthetic construct
EVQLLESGGGLVQPGGSLRLSCAASGFTFSTYAMSWVRQAPGKGLEWVSTIGPSGSSTYYAYADSVKGRFTISRDNSKNTLYLQMNSLRAEDTAVYYCAKEGPYSGYFTFDYWGQGTLVTVSS

SEQ ID NO: 7 - protein, 123 aa, Synthetic construct
EVQLLESGGGLVQPGGSLRLSCAASGFTFSTYAMSWVRQAPGKGLEWVSTIGPSGSSTYYAYADSVKGRFTISRDNSKNTLYLQMNSLRAEDTAVYYCAKESPYSAYFTFDYWGQGTLVTVSS

SEQ ID NO: 8 - protein, 121 aa, Synthetic construct
EVQLLESGGGLVQPGGSLRLSCAASGFTFSTYAMSWVRQAPGKGLEWVSTIGPSGSSTYYADSVKGRFTISRDNSKNTLYLQMNSLRAEDTAVYYCAKEGPNSGYFDFDYWGQGTLVTVSS

SEQ ID NO: 9 - protein, 121 aa, Synthetic construct
EVQLLESGGGLVQPGGSLRLSCAASGFTFSTYAMSWVRQAPGKGLEWVSTIGPSGSSTYYADSVKGRFTISRDNSKNTLYLQMNSLRAEDTAVYYCAKESPFSAYFTFDYWGQGTLVTVSS

SEQ ID NO: 10 - protein, 121 aa, Synthetic construct
EVQLLESGGGLVQPGGSLRLSCAASGFTFSTYAMSWVRQAPGKGLEWVSTIGPSGSSTYYADSVKGRFTISRDNSKNTLYLQMNSLRAEDTAVYYCAKEGPNSGYFTFDYWGQGTLVTVSS

SEQ ID NO: 11 - protein, 121 aa, Synthetic construct
EVQLLESGGGLVQPGGSLRLSCAASGFTFSTYAMSWVRQAPGKGLEWVSTIGPSGSSTYYADSVKGRFTISRDNSKNTLYLQMNSLRAEDTAVYYCAKEGPFSGYFTFDYWGQGTLVTVSS

SEQ ID NO: 12 - protein, 121 aa, Synthetic construct
EVQLLESGGGLVQPGGSLRLSCAASGFTFSTYAMSWVRQAPGKGLEWVSTIGPSGSSTYYADSVKGRFTISRDNSKNTLYLQMNSLRAEDTAVYYCAKEGPYSGYFTFDYWGQGTLVTVSS

SEQ ID NO: 13 - protein, 121 aa, Synthetic construct
EVQLLESGGGLVQPGGSLRLSCAASGFTFSTYAMSWVRQAPGKGLEWVSTIGPSGSSTYYADSVKGRFTISRDNSKNTLYLQMNSLRAEDTAVYYCAKESPYSAYFTFDYWGQGTLVTVSS

SEQ ID NO: 14 - protein, 107 aa, Synthetic construct
EIVLTQSPSSLSASVGDRVTITCRASQSISTYLNWYQQKPGKAPKVLIYAASSLQSGVPSRISGSGSGTDFTLTISSLQPEDFATYYCQQSYSIPLTFGGGTKLEIK

SEQ ID NO: 15 - protein, 107 aa, Synthetic construct
DIQMTQSPSSLSASVGDRVTITCRASQSISTYLNWYQQKPGKAPKLLIYAASSLQSGVPSRFSGSGSGTDFTLTISSLQPEDFATYYCQQSYSIPLTFGQGTKVEIK

SEQ ID NO: 16 - protein, 107 aa, Synthetic construct
DIQMTQSPSSLSASVGDRVTITCRASQSISTYLNWYQQKPGKAPKLLIYAASSLQSGVPSRFSGSGSGTDFTLTISSLQPEDFATYYCQQSYSRPLTFGQGTKVEIK

SEQ ID NO: 17 - protein, 107 aa, Synthetic construct
DIQMTQSPSSLSASVGDRVTITCRASQSISTYLNWYQQKPGKAPKLLIYAASSLQSGVPSRFSGSGSGTDFTLTISSLQPEDFATYYCQQSYSPPLTFGQGTKVEIK

SEQ ID NO: 18 - protein, 13 aa, Synthetic construct
AASGFTFSTYAMS

SEQ ID NO: 19 - protein, 10 aa, Synthetic construct
TIGPSGSSTY

SEQ ID NO: 20 - protein, 14 aa, Synthetic construct
AKEGPNSGYFDFDY

SEQ ID NO: 21 - protein, 14 aa, Synthetic construct
AKESPFSAYFTFDY

SEQ ID NO: 22 - protein, 14 aa, Synthetic construct
AKEGPNSGYFTFDY

SEQ ID NO: 23 - protein, 14 aa, Synthetic construct
AKEGPFSGYFTFDY

SEQ ID NO: 24 - protein, 14 aa, Synthetic construct
AKEGPYSGYFTFDY

SEQ ID NO: 25 - protein, 14 aa, Synthetic construct
AKESPYSAYFTFDY

SEQ ID NO: 26 - protein, 11 aa, Synthetic construct
RASQSISTYLN

SEQ ID NO: 27 - protein, 8 aa, Synthetic construct
YAASSLQS

SEQ ID NO: 28 - protein, 9 aa, Synthetic construct
QQSYSIPLT

SEQ ID NO: 29 - protein, 9 aa, Synthetic construct
QQSYSRPLT

SEQ ID NO: 30 - protein, 9 aa, Synthetic construct
QQSYSPPLT

SEQ ID NO: 31 - DNA, 20 bp, Synthetic construct
  source 1..20: mol_type = other DNA
aacccagacacaagcattcc

SEQ ID NO: 32 - DNA, 20 bp, Synthetic construct
  source 1..20: mol_type = other DNA
  modified_base 14: mod_base = i
gcctttgaggtttntggtca

SEQ ID NO: 33 - DNA, 24 bp, Synthetic construct
  source 1..24: mol_type = other DNA
  modified_base 18: mod_base = i
gaatggcagcacgctatnaaatcc

SEQ ID NO: 34 - DNA, 24 bp, Synthetic construct
  source 1..24: mol_type = other DNA
  modified_base 12: mod_base = i
  modified_base 22: mod_base = i
gccgctagaatncaaaacagtngg

SEQ ID NO: 35 - DNA, 20 bp, Synthetic construct
  source 1..20: mol_type = other DNA
gggacccgctgtcttctagt

SEQ ID NO: 36 - DNA, 22 bp, Synthetic construct
  source 1..22: mol_type = other DNA
tcaactcaaattcgctgaggac

SEQ ID NO: 37 - protein, 19 aa, Synthetic construct
CTGACCTCACAGATGCCAA

SEQ ID NO: 38 - protein, 20 aa, Synthetic construct
GGTCTGATAGTCTGTCACAA

SEQ ID NO: 39 - protein, 20 aa, Synthetic construct
GGCCTCACCCCATTTGATGT

SEQ ID NO: 40 - protein, 23 aa, Synthetic construct
CATGTTCCAGTATGACTCCACTC